(19) United States
(12) Patent Application Publication
(10) Pub. No.: US 2007/0248607 A1
(43) Pub. Date: Oct. 25, 2007

(54) NEGATIVE IMMUNOMODULATION OF IMMUNE RESPONSES BY NKG2D-POSITIVE CD4+ CELLS

(76) Inventors: Thomas Spies, Seattle, WA (US); Veronika Spies, Seattle, WA (US)

Correspondence Address: FULBRIGHT & JAWORSKI L.L.P., 600 CONGRESS AVE., SUITE 2400, AUSTIN, TX, 78701, US

(21) Appl. No.: 11/553,327

(22) Filed: Oct. 26, 2006

Related U.S. Application Data
(60) Provisional application No. 60/733,312, filed on Nov. 3, 2005.

(51) Int. Cl. A61K 39/395; A61K 38/00; A61K 38/16; C12N 5/00; G01N 33/53; C12Q 1/68; A61P 37/00; A61K 39/00

(52) U.S. Cl. 424/141.1; 424/130.1; 435/375; 435/6; 435/7.1; 514/12; 514/789; 514/8

(57) ABSTRACT

The present invention relates to methods of treating immune disorders, particularly autoimmune diseases and cancers having an immune-deficient aspect involving NKG2D-positive CD4⁺ cells. In addition, the invention provides screening methods that assess the ability of compositions to modulate the negative immunomodulatory effects of NKG2D-positive CD4⁺ cells. Also provided is a new target for inhibition of signaling by soluble MIC ligand, namely, the ERP-5 disulphide isomerase that interacts with MICA/MICB on the surface of tumor cells.

This application claims benefit of priority to U.S. Provisional Application Ser. No. 60/733,312, filed Nov. 3, 2005, the entire contents of which are hereby incorporated by reference.

### FIELD OF THE INVENTION

The present invention relates to methods of treating disorders having an aberrant immune response component, including cancers and autoimmune disorders such as rheumatoid arthritis. Also described are methods of screening for immunomodulatory agents that act through previously unknown immune regulating pathways, in particular, through an atypical NKG2D-positive population of CD4⁺ cells. In addition, methods for screening and treatment of MIC-associated diseases, targeting the interaction between ERP-5 and MICA/MICB, are provided.

### BACKGROUND

Maintaining effective immune surveillance without provoking autoimmune reactions requires the precise titration of effector T cell responses. This fine-tuning may involve the integration of negative or positive signals transduced by inhibitory or activating isoforms, such as the different killer cell Ig-like receptors (KIR), which interact with MHC class I HLA-A, -B, or -C alleles, and the inhibitory CD94-NKG2A and activating CD94-NKG2C heterodimers, which interact with HLA-E. Some of these receptors have the capacity to modulate thresholds of T cell antigen receptor-dependent T cell activation. In the rare absence of inhibitory receptors, the activating isoforms may augment T cell effector functions and contribute to autoimmune pathology.

NKG2D is an activating receptor that interacts with the MHC class I-related MICA and MICB glycoproteins, among other ligands. MICA and MICB have no role in antigen presentation, are generally only found in intestinal epithelium, and can be stress-induced in permissive types of cells by viral and bacterial infections, malignant transformation, and proliferation. NKG2D is a C-type lectin-like activating receptor that signals through the associated DAP10 adaptor protein, which is similar to CD28. Ligand engagement of NKG2D activates NK cells and potently co-stimulates effector T cells. However, expression of NKG2D is controlled by ligand-induced down-modulation, which is transient and rapidly reversed by T cell receptor stimulation and in the presence of IL-15.

NKG2D is expressed on most natural killer (NK) cells, CD8⁺ T cells, but not in general on CD4⁺ T cells. One exception to this rule is seen in rheumatoid arthritis, where the severity of autoimmune and inflammatory joint disease correlates with large numbers of autoreactive CD4⁺CD28⁻T cells that express NKG2D, which are scarce in healthy individuals (Groh et al., 2003). These T cells, found in peripheral blood and synovial tissue of RA patients, are stimulated by the stress-inducible MIC ligands of NKG2D, which are produced by RA synoviocytes, resulting in aberrant cytokine and proliferative responses. These results suggest that a profound lack of regulation of NKG2D and its MIC ligands may cause autoreactive T cell stimulation, thus promoting the self-perpetuating pathology in RA and possibly other autoimmune diseases (Groh et al., 2003). However, the full role of NKG2D-positive CD4⁺ cells in immune dysfunction is not fully understood.

### SUMMARY OF THE INVENTION

Thus, in accordance with the present invention, there is provided a method of screening for an immunomodulatory agent comprising (a) providing a cell that expresses ERP5 and MICA/MICB; (b) culturing the cell with a candidate substance; and (c) assessing one or more of (i) cell surface expression of MICA/MICB, (ii) MICA/MICB complexing with ERP5, (iii) disulfide bond reduction in MICA/MICB, (iv) MICA/MICB tetramer binding, (v) proteolytic cleavage of MICA/MICB, (vi) presence of soluble MICA/MICB, (vii) ERP5 transcription, translation or cell surface expression, and/or (viii) MICA/MICB autoantibodies, wherein a change in any of (c)(i)-(viii), as compared to a cell not treated with the candidate substance, identifies the candidate substance as an immunomodulatory agent. The candidate substance may be a peptide, protein, an RNA, a DNA, an organopharmaceutical, or a lipid. The cell may be an epithelial tumor cell, activated lymphocyte, synoviocyte, leukemia cell, activated hematopoietic cell, inflamed cell, infected cell or a cell derived from an autoimmune lesion.

In another embodiment, there is provided a method of modulating MICA/MICB cleavage in a cell that expresses MICA/MICB and ERP5 comprising contacting the cell with a modulator of ERP5 expression or function. The modulator may alter ERP5 release of MICA/MICB, alter ERP5 binding to MICA/MICB, alter ERP5 isomerization of MICA/MICB, alter ERP5 thioreduction of MICA/MICB, or alter ERP5 transcription or translation or cell surface expression. The cell may be an epithelial tumor cell, activated lymphocyte, synoviocyte, leukemia cell, activated hematopoietic cell, inflamed cell, infected cell or a cell derived from an autoimmune lesion. The modulator may be an antagonist that is a competing substrate for EPR5, such as a MIC fragment (e.g., the α3 domain of MICA or MICB), an antagonist that is a thioreductase inhibitor, or an antagonist selected from bacitracin, DTNB and PAO. The cell may be in a subject, such as an animal or human subject. The subject may have or be suspected of having cancer, an autoimmune disease, an inflammatory disease or an infection.

In still yet an additional embodiment, there is provided a method of modulating MICA/MICB cleavage in a cell that expresses MICA/MICB and ERP5 comprising contacting the cell with a modulator of ERP5 expression or function. The modulator may alter ERP5 release of MICA/MICB, alter ERP5 binding to MICA/MICB, alter ERP5 isomerization of MICA/MICB, alter ERP5 thioreduction of MICA/MICB, or alter ERP5 transcription or translation or cell surface expression. The cell may be an epithelial tumor cell, activated lymphocyte, synoviocyte, leukemia cell, activated hematopoietic cell, inflamed cell, infected cell or a cell derived from an autoimmune lesion. The modulator may be an antagonist that is a competing substrate for EPR5, or a thioreductase inhibitor, or be selected from bacitracin, DTNB and PAO.

A further embodiment comprises a method of screening for an agent that modulates the interaction of ERP5 and MICA/MICB comprising (a) providing isolated ERP5 or MICA/MICB-binding fragment thereof; (b) contacting ERP5 with MICA/MICB or an ERP5-binding fragment thereof in the presence of a candidate substance; and (c) assessing binding of ERP5 to MICA/MICB, wherein altered binding of ERP5 to MICA/MICB, as compared to binding in the absence of the candidate substance, indicates that the candidate substance is an agent that modulates the interaction of ERP5 and MICA/MICB comprising. The candidate substance may be a peptide, protein, an RNA, a DNA, an organopharmaceutical, or a lipid. The candidate substance may be MICA, MICB, anti-NKG2D antibodies, or anti-NKG2D antibody derivatives.

An additional embodiment comprises a method of characterizing a T cell population in a subject comprising (a) identifying a CD4⁺ population in a subject; and (b) assessing the relative proportions of NKG2D⁺ and NKG2D⁻ cells in the CD4⁺ population. The subject may be known to have an autoimmune disease or cancer, or suspected of having an autoimmune disease or cancer. The cancer may be a primary, metastatic, recurrent or drug resistant cancer. The higher the NKG2D⁺/NKG2D⁻ ratio, the more severe the cancer. The higher the NKG2D⁺/NKG2D ratio, the more likely the cancer will progress. The lower the NKG2D⁺/NKG2D⁻ ratio, the more severe the autoimmune disease. The lower the NKG2D⁺/NKG2D⁻ ratio, the more likely the autoimmune disease will progress. The method may further comprise assessing levels of MICA/MICB expression in the subject, assessing soluble MICA/MICB in the subject, assessing levels of FasL in the subject, or assessing levels of cytokines in the subject.

In another embodiment, there is provided a method of screening for an immunomodulatory agent comprising (a) providing (i) a population of NKG2D⁺ CD4⁺ cells, wherein the NKG2D⁺ CD4+ cells are stimulated with a NKG2D ligand, and (ii) a population of NKG2D⁻ CD4⁺ cells; (b) culturing together the NKG2D⁺ CD4⁺ cells, NKG2D⁻ CD4⁺ cells and a candidate substance; (c) assessing the survival, proliferation or activity of the NKG2D⁻ CD4⁺ cell population, wherein a change in the survival, proliferation or activity of the NKG2D⁻ CD4⁺ cells, as compared to NKG2D⁺ CD4⁺ cells cultured with NKG2D⁺ CD4⁺ cells in the absence of the candidate substance, indicates that the candidate substance is an immunomodulatory agent. The candidate substance is a peptide, a protein, an RNA, a DNA, an organopharmaceutical or a lipid. The method may further comprise a negative control, wherein a second population of NKG2D⁺ CD4⁺ cells is not stimulated with a NKG2D ligand prior to culturing with the candidate substance and the second population of NKG2D⁻ CD4⁺ cells. The NKG2D ligand may be MICA, MICB, anti-NKG2D antibodies, ULB1-ULB10, DAP10, or anti-NKG2D antibody derivatives. The activity of the NKG2D⁻ CD4⁺ cells may be cytokine production, apoptosis, growth arrest, cell cycle arrest, cell proliferation or expression of soluble mediators.

In another embodiment, there is provided a method of screening for an immunomodulatory agent comprising (a) providing (i) a population of NKG2D⁺ CD4⁺ cells, wherein the NKG2D⁺ CD4⁺ cells are stimulated with a NKG2D ligand, and (ii) a population of NKG2D⁻ CD4⁺ cells; (b) culturing the NKG2D⁻ CD4⁺ cells in the presence of a candidate substance; (c) culturing the NKG2D⁺ CD4⁺ cells or a cell supernatant therefrom with the cells of step (b); (d) assessing the survival, proliferation or activity of the NKG2D⁻ CD4⁺ cells, wherein a change in the survival, proliferation or activity of the NKG2D⁻ CD4⁺ cells, as compared to NKG2D CD4⁺ cells cultured in the absence of the candidate substance, indicates that the candidate substance is an immunomodulatory agent. The candidate substance may be a peptide, protein, an RNA, a DNA, an organopharmaceutical or a lipid. The method may further comprise a negative control, wherein a second population of NKG2D⁺ CD4⁺ cells is not stimulated with a NKG2D ligand prior to culturing with the candidate substance and the second population of NKG2D CD4⁺ cells. The NKG2D ligand may be MICA, MICB, anti-NKG2D antibodies, ULB1-ULB10, DAP10, or anti-NKG2D antibody derivatives. The activity of the NKG2D⁻ CD4⁺ cells may be cytokine production, apoptosis, growth arrest, cell cycle arrest, cell proliferation or expression of soluble mediators.

In yet another embodiment, there is provided a method of screening for an immunomodulatory agent comprising (a) providing (i) a population of NKG2D⁺ CD4⁺ cells, wherein the NKG2D⁺ CD4⁺ cells are stimulated with a NKG2D ligand, and (ii) a population of NKG2D-CD4+ cells; (b) culturing the NKG2D⁺ CD4⁺ cells in the presence of a candidate substance; (c) culturing the NKG2D CD4⁺ cells with the cells of step (b) or a cell supernatant of step (b); (d) assessing the survival, proliferation or activity of the NKG2D⁻ CD4⁺ cells, wherein a change in the survival, proliferation or activity of the NKG2D⁻ CD4⁺ cells, as compared to NKG2D⁻ CD4⁺ cells cultured with NKG2D⁺ CD4⁺ cells or supernatant cultured in the absence of the candidate substance, indicates that the candidate substance is an immunomodulatory agent. The method may further comprise a negative control, wherein a second population of NKG2D⁺ CD4⁺ cells is not stimulated with a NKG2D ligand prior to culturing with the candidate substance and the second population of NKG2D⁻ CD4⁺ cells. The NKG2D ligand may be MICA, MICB, anti-NKG2D antibodies, ULB1-ULB10, DAP10, or anti-NKG2D antibody derivatives. The activity of the NKG2D⁻ CD4⁺ cells may be cytokine production, apoptosis, growth arrest, cell cycle arrest, cell proliferation or expression of soluble mediators.

In still yet another embodiment, there is provided a method of screening for an immunomodulatory agent comprising (a) providing a population of NKG2D⁺ CD4⁺ cells, wherein the NKG2D⁺ CD4⁺ cells are stimulated with a NKG2D ligand; (b) culturing the NKG2D⁺ CD4⁺ cells with a candidate substance; and (c) assessing the production and/or secretion of a soluble mediator by the NKG2D⁺ CD4⁺ cells, wherein a change in the soluble mediator production and/or secretion by the NKG2D⁺ CD4⁺ cells, as compared to NKG2D⁺ CD4⁺ cells not treated with the candidate substance, identifies the candidate substances an immunomodulatory agent. The candidate substance may be a peptide, a protein, an RNA, a DNA, an organopharmaceutical or a lipid. The soluble mediator may be FasL. The step of assessing may comprise anti-FasL ELISA, FasL-based FACS, HPLC, FPLC, protein analysis, MS, electrophoresis, Western blotting, expression analysis, PCR, Southern blotting, Northern blotting, microarray analysis, or determining Jurkat cell apoptosis by the supernatant of step (b). The NKG2D ligand may be MICA, MICB, an anti-NKG2D antibody, or an anti-NKG2D antibody derivative.

In a further embodiment, there is provided a method of modulating production of soluble factors by an NKG2D⁺ CD4⁺ cell comprising administering to the cell a soluble MIC or MIC fragment or other NKG2D antagonist such as an NKG2D⁺ antibody or antibody derivative. The factor being modulated may comprise a cytokine or FasL. The method may further inhibit NKG2D signaling, cell growth, cell proliferation or may promote cell death. This regulation may be antigen-dependent or independent.

In a further embodiment, there is provided a method of treating a subject with an epithelial cell-derived tumor comprising administering to the subject an antagonist of an NKG2D⁺ CD4⁺ cell. The antagonist may be an inhibitory NKG2D⁺ antibody or antibody derivative. The antagonist may inhibit NKG2D signaling, expression of a soluble mediator, secretion of a soluble mediator, cell growth, or cell proliferation.

In yet a further embodiment, there is provided a method of treating a subject with an MIC-secreting tumor comprising (a) assessing MIC secretion by the tumor and (b) administering to the subject an antagonist of FasL. The antagonist may be anti-FasL antibody, a FasL siRNA, or a competitive inhibitor for FasL receptor binding. MIC secretion may be assessed by measuring MICA and/or MICB levels in blood or serum.

In still yet a further embodiment, there is provided a method of treating a subject with an autoimmune disease comprising administering to the subject an agonist of an NKG2D⁺ CD4⁺ cell. The agonist may be MICA, MICB, anti-NKG2D⁺ antibody or derivative thereof, DAP10 or ULB1-10. The autoimmune disease may be RA, SLE, juvenile SLE, sclerodema, MS, Crohn's disease, celiac disease, inflammatory bowel disease, rheumatoid arthritis, insulin-dependent diabetes mellitus (type 1), multiple sclerosis, Wegener's granulomatosis, Sjogren's syndrome, systemic lupus erythematosus, myasthenia gravis, Reiter's syndrome, Grave's disease, Hashimoto's thyroiditis, pernicious anemia, Addison's disease, dermatomyositis, polymyositis, T-cell mediated transplant rejection (e.g., GVHD) and Guillain Barré.

In an additional embodiment, there is provided a method of treating a subject with an autoimmune disease comprising administering to the subject an agonist of FasL activity. The autoimmune disease may be RA, SLE, juvenile SLE, sclerodema, MS, Crohn's disease, celiac disease, inflammatory bowel disease, rheumatoid arthritis, insulin-dependent diabetes mellitus (type 1), multiple sclerosis, Wegener's granulomatosis, Sjogren's syndrome, systemic lupus erythematosus, myasthenia gravis, Reiter's syndrome, Grave's disease, Hashimoto's thyroiditis, pernicious anemia, Addison's disease, dermatomyositis, polymyositis, T-cell mediated transplant rejection (e.g., GVHD) and Guillain Barré.

In another aspect, the present invention provides kits comprising any one or more of the herein-described compounds. Typically, the kit also comprises instructions for using the antibodies according to the present methods.

In another aspect, the present invention contemplates ex vivo methods of modulating MICA/MICB cleavage in a cell that expresses MICA/MICB and ERP5 comprising contacting the cell with a modulator of ERP5 expression or function. In yet another aspect, the present invention contemplates in vivo methods of modulating MICA/MICB cleavage in a non-human cell that expresses MICA/MICB and ERP5 comprising contacting the cell with a modulator of ERP5 expression or function. Also contemplated is the use of a binding agent capable of binding MICA/MICB and blocking cleavage of MICA and/or MICB by ERP5 in the manufacture of a pharmaceutical exerting its effect by reducing MICA and/or MICB cleavage by ERP5. In some embodiments, the use of an agonist of an NKG2D⁺ CD4⁺ cell in the manufacture of a pharmaceutical for the treatment of autoimmune diseases is contemplated. In certain embodiments, the use of an agonist of FasL activity in the manufacture of a pharmaceutical for the treatment of autoimmune diseases is contemplated.

It is contemplated that any method or composition described herein can be implemented with respect to any other method or composition described herein.

The use of the word, “a” or “an” when used with the term “comprising” in the specification and/or claims may mean “one,” “one or more,” “at least one,” or “one or more than one.”

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1—Expansion of NKG2D⁺ CD4⁺ T cells in patients with MIC-positive tumors. TIL, tumor-infiltrating lymphocytes; PBMC, peripheral blood mononuclear cells. Only about 0.5-2% of normal CD4 T cells are positive for NKG2D (not shown). BT, breast tumor; OT, ovarian tumor; LT, lung tumor.

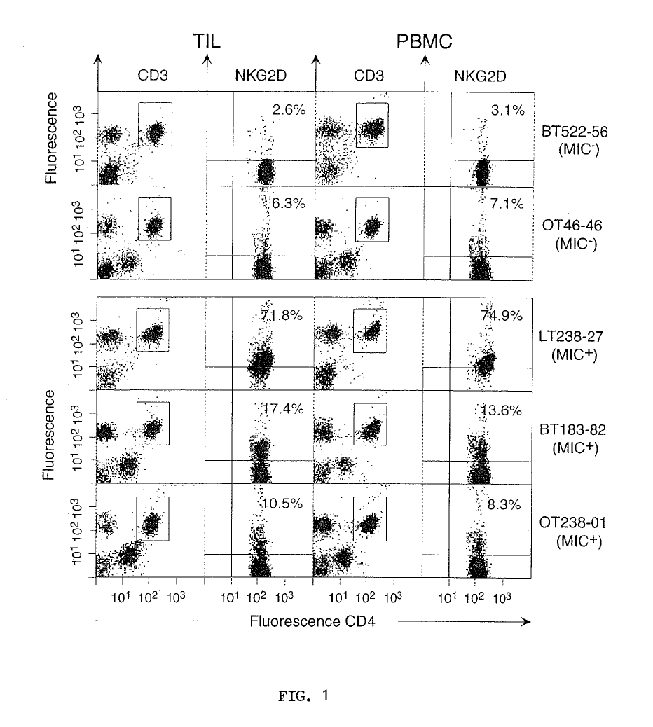

FIG. 2—Ranges of NKG2D mean fluorescence on controls and TIL and PBMC from MIC− and MIC+ tumor patients. N, sample numbers.

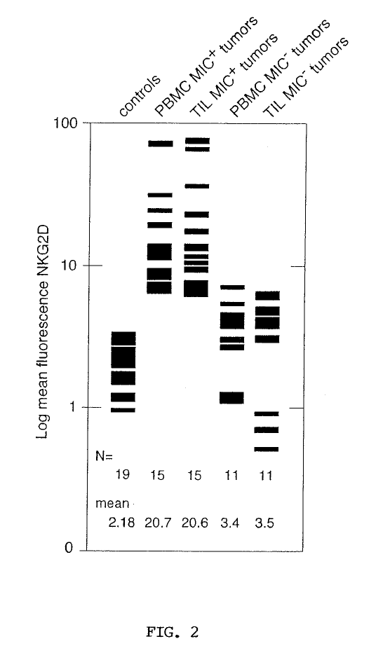

FIG. 3—NKG2D⁺ CD4⁺ T cells express memory (CD45RO) and activation (CD25, HLA-DR) markers.

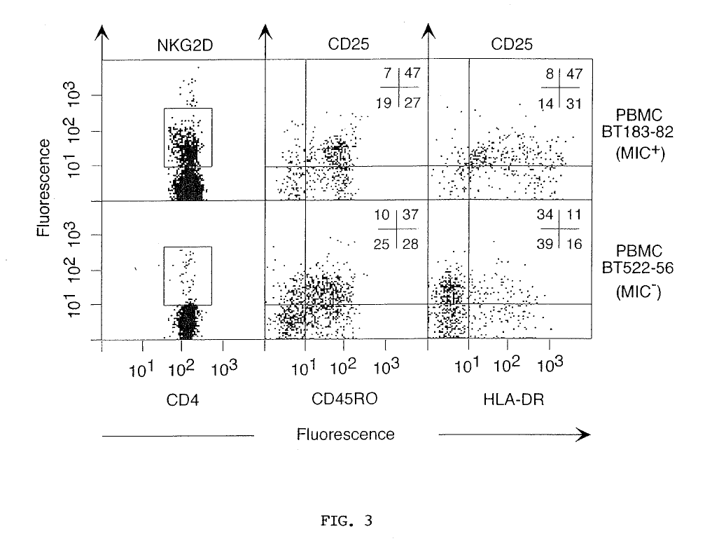

FIG. 4—NKG2D Induction on normal CD4⁺ T cells upon anti-CD3 activation. Induction is enhanced in the additional presence of soluble MICA (sMICA) as a result of costimulation.

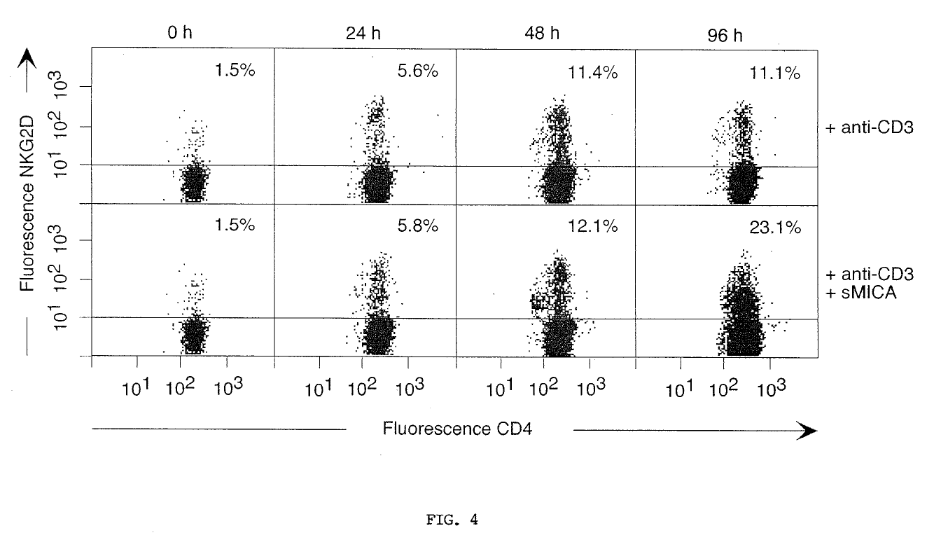

FIGS. 5A-D—(FIG. 5A) Ligand (sMICA)-induced expansion of NKG2D⁺ CD4⁺ T cells (see percent numbers) and inhibition of NKG2D⁻ CD4⁺ T cell proliferation as shown by CFSE dilution. (FIG. 5B) Dose-dependent inhibition of proliferation. (FIGS. 5C and D) Propidium iodide (PD stainings reveal partial cell cycle arrests in G0/G1 after exposure to sMICA or anti-NKG2D antibody.

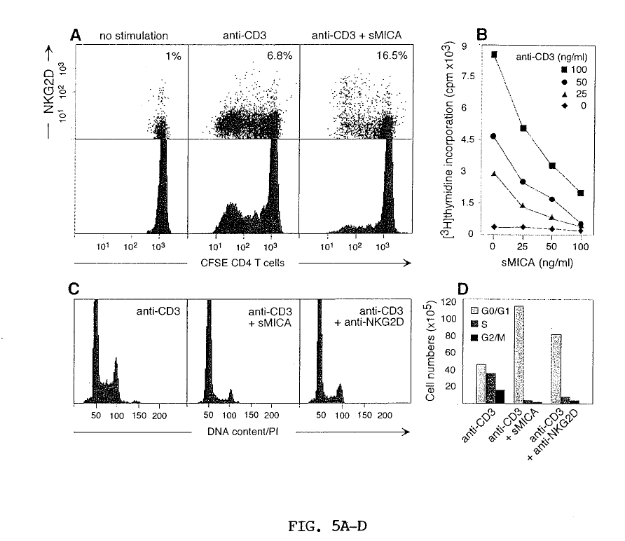

FIGS. 6A-B—(FIG. 6A) Inhibition of CD4 T cell proliferation by sMICA, tumor patient serum BT450-85 (and neutralization by anti-MIC mAb 6D4; by MICA and ULBP1 transfectants of C1R cells, and by anti-NKG2D. (FIG. 6B) Kinetics of NKG2D induction during time course of sMICA addition.

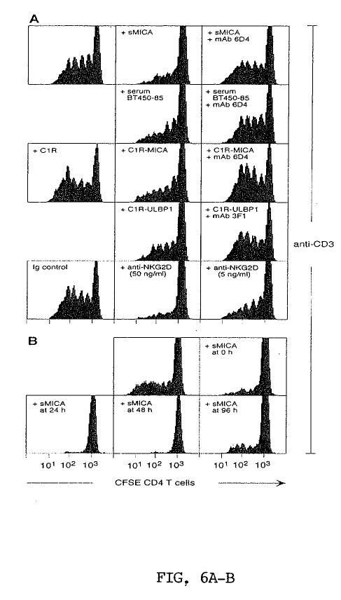

FIG. 7-Transwell and T cell depletion experiments demonstrate that suppression of T cell proliferation is independent of cell-cell contact and is mediated by a soluble factor that is produced by NKG2D⁺ but not by NKG2D⁻ CD4⁺ T cells.

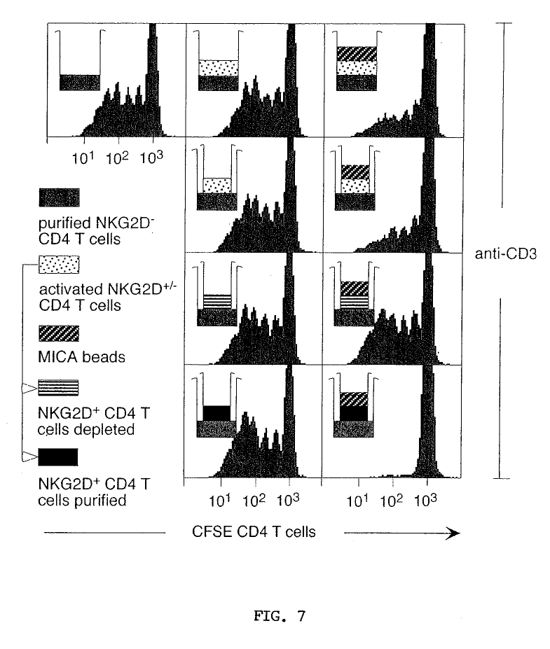

FIGS. 8A-D—(FIG. 8A) Cytokine and Fas-L secretion profiles of anti-CD3, or anti-CD3 plus sMICA or anti-NKG2D stimulated CD4⁺ T cells. (FIG. 8B) Surface detection of Fas-L on NKG2D⁺ but not on NKG2D⁻ CD4⁺ T cells. (FIG. 8C) Supernatant from cultured CD4⁺ T cells activated with anti-CD3 and sMICA induce Jurkat cell apoptosis (center dot plot) similar to activating anti-Fas antibody (left dot plot). This activity can be inhibited by a blocking anti-Fas antibody (right dot plot). (FIG. 8D) Abrogation of inhibition of T cell proliferation by a blocking anti-Fas antibody.

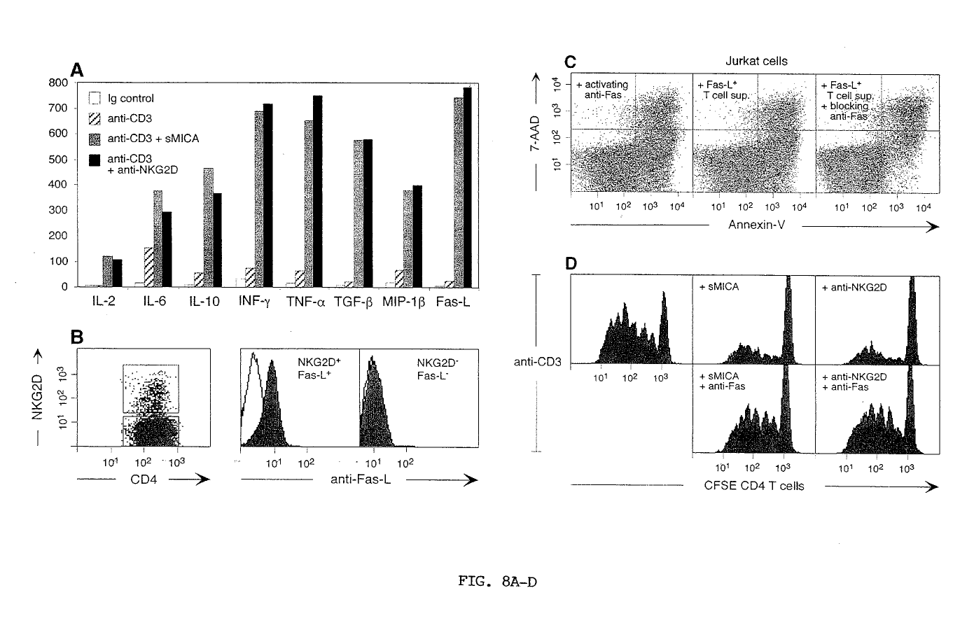

FIGS. 9A-B—Requirement of tumor cell surface-associated thioreductase activity for MICA shedding. (FIG. 9A) Inhibition of sMICA shedding by thioreductase inhibitors bacitracin, DTNB [dithiobis(2-nitrobenzoic acid)] and PAO (phenyl arsenic oxide). (FIG. 9B) PAO inhibits MICA tetramer binding to tumor cell lines.

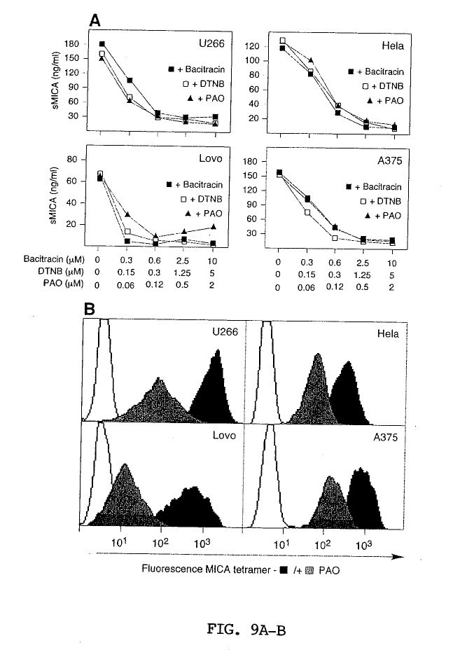

FIGS. 10A-C—Specific MICA tetramer binding independent of NKG2D and tentative surface interactions of MICA with ERP5 and GRP78. (FIG. 10A) Flow cytometry analysis confirmed binding of MICA (filled profiles) and ULBP (lightly shaded profiles) tetramers to NKL cells and complete inhibition of binding by anti-NKG2D mAb 1D11 (shaded profiles). MICA but not ULBP2 tetramers bind to NKG2D⁻ U266, Hela, HCT116, Lovo, and A375 tumor cells and binding is inhibited by recombinant soluble MICA (rsMICA; shaded profiles) produced in bacteria. U937 cells are negative for MICA tetramer binding. Open profiles in all panels represent IgG control stainings. (FIG. 10B) Silver staining after SDS-PAGE of U266 and U937 outer cell membrane proteins enriched for binding to MICA beads. TRX, thioredoxin. (FIG. 10C) Probing of MICA bead-purified proteins from surface biotinylated cells after SDS-PAGE and immunoblotting with streptavidin-HRP or specific antisera confirms surface localization of GRP78 and ERP5. The two minor additional bands in the anti-ERP5 panel likely are related proteins that are cross-reactive with the antiserum.

FIGS. 10A-C—Tumor-associated ERP5 surface expression and pharmacological inhibition of sMICA shedding. (FIG. 11A) Freshly isolated breast, ovarian, and melanoma tumor cells show similar patterns of MICA expression and fluorescence intensities of MICA tetramer binding. (FIG. 11B) Thioreductase inhibitors bacitracin, DTNB, and PAO reduce shedding of sMICA by Hela and A375 cells in a dose-dependent manner as determined by ELISA. Data shown are representative of three experiments. (FIG. 11C) PAO interferes with MICA tetramer binding.

FIGS. 12A-C—ERP5 is required for sMICA shedding. (FIG. 12A) Expression of siRNA constructs 17 or 19 by retroviral transduction of A375 cells results in 70-80% reductions of cellular ERP5 mRNA as measured by real-time RT-PCR. (FIG. 12B) Knock-down of ERP5 mRNA decreases MICA tetramer binding (right column, filled profiles); open profiles represent mock-transduced cells. MICA expression (left column, shaded profiles) is unchanged; open profiles represent IgG control stainings. (FIG. 12C) Knock-down of ERP5 mRNA diminishes sMICA shedding.

FIGS. 13A-B—ERP5-MICA disulfide exchange enables cleavage of membrane-anchored MICA. (FIG. 13A) Treatment of Hela cells with TCA before immunoprecipitation with anti-MICA from lysates of surface-biotinylated cells, N-glycanase-mediated protein deglycosylation, non-reducing SDS-PAGE, and immunoblotting reveals MICA-ERP5 complexes (lane 1). Protein identities are confirmed by secondary precipitations (lanes 7, 8 and 10). Additional controls are provided by primary precipitations of ERP5 (lanes 5 and 6) and by purified rERP5 (lane 11). After cell culture in the presence of increasing concentrations of sRNase, the amounts of co-immunoprecipitating ERP5 increase (lanes 1-4), full-length MICA disappears (compare lanes 1 and 2 with lanes 3 and 4), and sMICA emerges (lanes 3 and 4). The identity of sMICA is confirmed by secondary precipitation (lane 9) and comparison to sMICA isolated from cell culture media (lane 12). (FIG. 13B) Increased concentrations of sRNase promote sMICA shedding by A375 and Hela cells. Data shown are representative of three experiments.

FIGS. 14A-C—Specific reduction of MICA by ERP5 in vitro. (FIG. 14A) ERP5 (lane 1) and MICA (lane 2) run at their expected molecular weights when analysed under reducing conditions (+β-NE) by SDS-PAGE. Unreduced MICA (lane 3) displays increased electrophoretic mobility consistent with stable intrachain disulfide linkages. Co-incubation of MICA and ERP5 for 1 h (lane 4) or 3 h (lane 5) at room temperature reveals progressive MICA reduction. (FIGS. 14B-C) No reduction by ERP5 is apparent of Siderocalin and CD94-NKG2A.

FIGS. 15A-E—ERP5 substrate specificity for the membrane-proximal α3 domain of MICA. (FIG. 15A) Schematic representation of ERP5 domain structure showing active site CGHC motifs within thioredoxin domains (open boxes). Upper numbers refer to amino acid positions at domain boundaries; lower numbers identify positions of cysteine residues and internal truncation sites of the separately expressed ERP5 fragments. The KDEL sequence represents the C-terminal endoplasmic reticulum retention motif. (FIG. 15B) Non-reducing SDS-PAGE shows that MICA is partially reduced (lane 3) by the N-terminal ERP5 Filled and open arrow heads on the right of all panels indicate positions of non-reduced and reduced forms, respectively, of MICA substrate and control proteins.polypeptide fragment including amino acid residues 1-118. (FIG. 15C) Incubation of MICA substrate with the active site C39S mutant of ERP5 fragment 1-118 for 1 or 3 h (lanes 5 and 6) results in disulfide-linked heterodimers (position marked by asterisk) which are resolved by β-ME (lane 7). Mutant ERP5 fragments partially form homodimers under non-reducing conditions (compare lane 3 with lanes 4-6). (FIG. 15D) ERP5 has no effect on the MICA α1α2 platform domain. Partial reduction in lane 2 is due to bleeding of reducing agent from lane 1. (FIG. 15E) The ERP5 1-118 C39S mutant polypeptide reduces the α3 domain disulfide bond of MICA as indicated by the occurrence of unresolved protein heterodimers (lanes 4 and 5; position marked by two asterisks). As in FIG. 15C, the mutated ERP5 fragments partially form homodimers. Filled and open arrow heads on the right of FIGS. 15B-D indicate positions of non-reduced and reduced forms, respectively, of MICA substrate proteins.

### DETAILED DESCRIPTION OF THE INVENTION

The present invention addresses the interaction of MIC ligands with ERP-5, an endoplasmic reticulum protein of the protein disulphide isomerase (PDI) family with which MICA specifically associates on the surface of tumor cells. Pharmacological inhibition of thioreductase catalytic activities and siRNA-mediated silencing of ERP5 expression profoundly reduce shedding of soluble MICA and interfere with its physical interaction with ERP5. The inventors also show that ERP-5 reduction of an intradomain disulphide bond of MICA causes a conformational destabilization that is a necessary prerequisite for proteolytic cleavage (by a distinct proteolytic entity) within the peptide sequence that connects the proximal α3 and transmembrane domains. Targeting this interaction provides a new mechanism for screening of drugs and for therapeutic intervention in MIC-related disease states.

In another aspect of the invention, methods for screening of compositions for immunomodulatory effects through NKG2D-positive CD4⁺ cells, as well as treating relating disease states, are provided. Also provided are methods relating to screening and therapy of MIC-related pathologies by targeting the interaction between MIC ligands and the ERP-5 disulphide isomerase.

The present invention thus is based, in part, on the surprising discovery that NKG2D-positive CD4⁺ cells demonstrate a profound negative immunomodulation of NKG2D-negative CD4⁺ cells, which are needed for the generation of immune responses, such as those seen against tumor cells. Thus, by inhibiting this regulation in the cancer setting, one can diminish or prevent the immunosuppression observed in MIC-related cancers. In addition, this pathway may be exploited in a positive fashion to achieve down-regulation of the MIC-related autoimmune dysfunction, in contrast to previously suggested inhibition of these cells in rheumatoid arthritis (Groh et al., 2003; also U.S. Ser. No. 10/898,003 and PCT/US03/12299, incorporated herein by reference).

### I. DEFINITIONS

As used herein, the following terms have the meanings ascribed to them unless specified otherwise.

As used herein, “T cells” refers to a sub-population of lymphocytes that mature in the thymus, and which display, among other molecules, T cell receptors on their surface. T cells can be identified by virtue of certain characteristics and biological properties, such as the expression of specific surface antigens including the TCR, CD4 or CD8, the ability of certain T cells to kill tumor or infected cells, the ability of certain T cells to activate other cells of the immune system, and the ability to release protein molecules called cytokines or other soluble mediators that stimulate or inhibit the immune response. Any of these characteristics and activities can be used to identify T cells using methods well known in the art.

The term “NKG2D” refers to an activating cell surface molecule that is found consistently on all or a fraction of numerous types of immune cells, particularly NK cells, CD8⁺ T cells, some CD4⁺ T cells, and γ/δ T cells. NKG2D is also referred to as killer cell lectin-like receptor, subfamily C, member 4, or as KLRC4 (see, e.g., OMIM 602893, the entire disclosure of which is herein incorporated by reference in its entirety). As used herein NKG2D refers to any NKG2D isoform, e.g., the isoforms described in Diefenbach et al. (2002). In NK and T cells, NKG2D can form heterodimers with proteins such as DAP10 (see, e.g., OMIM 604089) or DAP12 (see, e.g., OMIM 604142). It will be appreciated that any activity attributed herein to NKG2D, e.g., cell activation, recognition by antibodies, etc., can also refer to NKG2D-including complexes such as NKG2D-DAP10 or NKG2D-DAP12 heterodimers.

“Autoimmune” disorders include any disorder, condition, or disease in which the immune system mounts a reaction against self cells or tissues, due to a breakdown in the ability to distinguish self from non-self or otherwise. Examples of autoimmune disorders include Hashimoto's thyroiditis, pernicious anemia, Addison's disease, type I diabetes, rheumatoid arthritis, systemic lupus erythematosus, dermatomyositis, Crohn's disease, celiac disease, inflammatory bowel disorder, Sjogren's syndrome, lupus erythematosus, multiple sclerosis, myasthenia gravis, Reiter's syndrome, Grave's disease, polymyositis, Guillain Barré, Wegener's granulomatosus, polyarteritis nodosa, polymyalgia rheumatica, temporal arthritis, Bechet's disease, Churg-Strauss syndrome, Takayasu's arteritis, and others. Autoimmune disorders can involve any component of the immune system, and can target any cell or tissue type in the body.

“Cancer” refers to any hyperproliferative disorder, but in particular, it refers to malignancies involving almost any tissue, including brain, head & neck, esophagus, mouth & gums, trachea, lung, stomach, colon, liver, pancreas, kidney, rectum, ovary, uterus, cervix, testes, prostate, bladder, penis, vagina or blood. Cancer also refers to cancers that are primary, metastatic, recurrent and drug resistant. An “epithelial cancer” is one that is derived from an epithelial tissue and may occur in any location of the body, including ovarian cancer, squamous cell carcinoma, thyroid cancer, mammary neoplasia, and basal cell carcinoma. A “MIC-related cancer” is one that is characterized by MIC production by the cancer cell/tumor.

As used herein, the term rheumatoid arthritis refers to any disorder involving inflammation of the joints, and including features such as joint erosion, lymphocyte infiltration, synovial hyperplasia, aggressive proliferation of fibroblast-like synoviocytes and macrophages, and/or the presence of CD4⁺NKG2D⁺ cells.

The terms “reducing,” “interfering,” “inhibiting,” “down-modulating,” “antagonize,” and “down-regulating,” with respect to NKG2D or NKG2D-expressing cells means a process, method, or compound that can slow, reduce, reverse, or in any way negatively affect the activity or number of NKG2D receptors or the number of cells expressing NKG2D. These terms can refer to compounds that inhibit the activation of NKG2D by a ligand, that act antagonistically in the absence of a ligand to decrease the activity of the receptor, that decrease the expression level of the receptor, that block NKG2D-triggered signaling and/or gene expression, or that block any other activity of the cell that results from NKG2D activation. In a particular embodiment, the inhibiting compound or method targets the binding of the receptor by a ligand, e.g., by binding to the receptor and preventing ligand access. Alternatively, the inhibiting compound may interference with the production, processing or secretion of MICA and/or MICB.

The term “antibody,” as used herein, refers to polyclonal and monoclonal antibodies. Depending on the type of constant domain in the heavy chains, antibodies are assigned to one of five major classes: IgA, IgD, IgE, IgG, and IgM. Several of these are further divided into subclasses or isotypes, such as IgG1, IgG2, IgG3, IgG4, and the like. An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids that is primarily responsible for antigen recognition. The terms variable light chain (V_{L}) and variable heavy chain (V_{H}) refer to these light and heavy chains respectively. The heavy-chain constant domains that correspond to the different classes of immunoglobulins are termed “alpha,” “delta,” “epsilon,” “gamma” and “mu,” respectively. The subunit structures and three-dimensional configurations of different classes of immunoglobulins are well known. IgG and/or IgM are the preferred classes of antibodies employed in this invention, with IgG being particularly preferred, because they are the most common antibodies in the physiological situation and because they are most easily made in a laboratory setting. Preferably the antibody of this invention is a monoclonal antibody. Particularly preferred are humanized, bispecific, chimeric, human, or otherwise-human-suitable antibodies. “Antibodies” also includes any fragment or derivative of any of the herein described antibodies.

The term “specifically binds to” means that a ligand that can bind preferably in a competitive binding assay to the binding partner, as assessed using either recombinant forms of the proteins, epitopes therein, or native proteins present on the surface of isolated T or NK or other target cells. Competitive binding assays and other methods for determining specific binding (e.g., antibody masking) are further described below and are well known in the art.

A “human-suitable” or “humanized” antibody refers to any antibody, derivatized antibody, or antibody fragment that can be safely used in humans for, e.g., the therapeutic methods described herein. Human-suitable antibodies include all types of chimeric or fully human antibodies, or any antibodies in which at least a portion of the antibodies is derived from humans or otherwise modified so as to avoid the immune response that is generally provoked when native non-human antibodies are used.

For the purposes of the present invention, a “humanized” antibody refers to an antibody in which the constant and variable framework region of one or more human immunoglobulins is fused with the binding region, e.g., the CDR, of an animal immunoglobulin. Such humanized antibodies are designed to maintain the binding specificity of the non-human antibody from which the binding regions are derived, but to avoid an immune reaction against the non-human antibody.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

A “human” antibody is an antibody obtained from transgenic mice or other animals that have been “engineered” to produce specific human antibodies in response to antigenic challenge (see, e.g., Green et al., 1994; Lonberg et al., 1994; Taylor et al., 1994, the entire teachings of which are herein incorporated by reference). A fully human antibody also can be constructed by genetic or chromosomal transfection methods, as well as phage display technology, all of which are known in the art (see, e.g. McCafferty et al., 1990). Human antibodies may also be generated by in vitro activated B cells (see, e.g., U.S. Pat. Nos. 5,567,610 and 5,229,275, which are incorporated in their entirety by reference).

Within the context of this invention, “active” or “activated” T cells designate biologically active T cells, more particularly T cells having the capacity of cytolysis or of stimulating an immune response by, e.g., secreting cytokines.

The terms “isolated”, “purified” or “biologically pure” refer to material that is substantially or essentially free from components which normally accompany it as found in its native state. Purity and homogeneity are typically determined using analytical chemistry techniques such as polyacrylamide gel electrophoresis or high performance liquid chromatography. A protein that is the predominant species present in a preparation is substantially purified.

The term “biological sample” as used herein includes but is not limited to a biological fluid (for example serum, lymph, blood), cell sample or tissue sample (for example bone marrow, tumor biopsy).

The terms “polypeptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (nonrecombinant) form of the cell or express native genes that are otherwise abnormally expressed, under expressed or not expressed at all.

### II. NKG2D

NKG2D, a homodimeric C-type lectin-like receptor, is a unique stimulatory molecule that is found on natural killer (NK) cells CD8 αβ T cells and γΔ T cells. It is associated with adaptor proteins, designated DAP10, through oppositely charged amino acid residues in their transmembrane domains. DAP10 signals similarly to the CD28 co-stimulatory receptor by activation of phosphatidylinositol 3-kinase (PI3K) upon phosphorylation of a YxxM motif in its cytoplasmic domain. The deglycosylated NKG2D polypeptide chain is of 28 kilodalton (kD). It is encoded by a gene in the NK complex (NKC) on human chromosome 12. Despite its name, NKG2D shares no significant sequence homology with the NKG2A and NKG2C/H receptors that associate with CD94. NKG2D homodimers form stable complexes with monomeric MICA in solution, indicating that no other components are required to facilitate this interaction. Soluble NKG2D also binds to cell surface MICB, which has structural and functional properties similar to those of MICA (Steinle et al., 2001).

The inventors previously have shown that NKG2D functions as a receptor for MICA and MICB using biochemical and genetic methods (Bauer et al., 1999). Prior to this finding, the function of NKG2D was unknown. The inventors determined that NKG2D has a very broad distribution on lymphocyte subsets, being expressed on most NK cells, CD8 α/β T cells and γ/δ T cells. Functional experiments showed that engagement of NKG2D activates cytolytic responses of γ-delta T cells and NK cells against transfectants and epithelial tumor cells expressing MIC (Groh et al., 1999; Bauer et al., 1999). These results define an activating immunoreceptor-MHC ligand interaction that may promote antitumor NK and T cell responses. Furthermore, the inventors showed that interactions of MIC with NKG2D potently augment cytolytic responses of antigen-specific CD8 α/β T cell responses and co-stimulate cytokine production and T cell proliferation (Groh et al., 2001).

An activating receptor lacking an apparent antagonist is NKG2D, which interacts with the MHC class I-related MICA and MICB glycoproteins (discussed below) among other ligands (Bauer et al., 1999). These have no role in antigen presentation, have a restricted tissue distribution in intestinal epithelium, and can be stress-induced in permissive types of cells by viral and bacterial infections, malignant transformation and proliferation (Groh et al., 1996; Groh et al., 1998; Das et al., 2001; Groh et al., 2001; Tieng et al., 2002). NKG2D is a C-type lectin-like activating receptor that signals through the associated DAP10 adaptor protein similar to CD28 (Wu et al., 1999). It is expressed on most NK cells, CD8 T cells and 76 T cells, but only a few CD4 T cells (Bauer et al., 1999). Ligand engagement of NKG2D activates NK cells and potently costimulates effector T cells (Bauer et al., 1999; Das et al., 2001; Groh et al., 2001). However, the expression of NKG2D is controlled by ligand-induced down-modulation, which is transient and rapidly reversed by interleukin-15 (Groh et al., 2002).

The present invention addresses, in one aspect, a particular subset of CD4⁺ T cells that express the NKVG2D receptor. In normal subject, the percentage of these cells in the overall CD4⁺ population is very low, on the order of about 2% or less. However, in certain subjects, such as those with autoimmune diseases and MIC-related cancers, the percentage is much higher, and in some instances the majority of CD4⁺ cells express the NKVG2D receptor. Thus, aspects of the invention deal with the identification, purification, activation and inhibition of NKVG2D⁺ CD4⁺ cells. These cells, which are one member of the “regulatory T cell” (Treg) class, are an important group of cells, the control of which can permit one to control effector cells which receive signals from Tregs like these.

### III. MIC AND MIC LIGANDS

A. MIC

The primary ligands for NKG2D are MICA and MICB, distant relatives of MHC class I molecules that play no role in antigen presentation. Rather, they function as signals of cellular distress. These proteins have a highly restricted tissue distribution in intestinal epithelium and are frequently expressed in epithelial tumors (Groh et al., 1996; Groh et al., 1999) and in synovial tissues of patients with rheumatoid arthritis (Groh et al., 2003).

MICA and MICB proteins (SEQ ID NO: 2 and SEQ ID NO: 4, respectively) are MHC class I related Chains A and B. They are closely related and are encoded by genes 40 and 110 kilobases (kb) centromeric of HLA-B, respectively (Bahram et al., 1994). Sequences directly homologous to MIC are conserved in most mammals except rodents, and thus probably originated at an early stage in mammalian evolution. The translation product of MICA is only distantly similar to mammalian MHC class I chains, but it shares the same domain organization and predictably a similar tertiary structure. An average of 25% of the MICA amino acids in the extracellular α1, α2, and α3 domains match residues in diverse human and mouse, or in any other mammalian MIC class I sequences (Bahram et al., 1994). A further characteristic of MICA is the complete absence of all of the residues implicated in the binding of CD8 and the presence of eight N-linked glycosylation sites in the α1-α3 domain sequences. Moreover, transcription of MICA is restricted to various epithelial cell lines and is not regulated by γ-interferon. MICB mRNA is present in the same cell lines, albeit at very low levels. In epithelial cell lines, transcription of both MICA and MICB can be induced by heat shock in a manner similar to heat shock protein 70 (hsp70), presumably owing to the presence of putative heat shock elements (HSE) in the 5′ flanking regions of both MICA and MICB (Groh et al., 1996; Groh et al, 1998). Because of this property, MICA and MICB are cell stress response genes.

The inventors have previously reported the complete nucleotide sequence of the MICA gene comprising 11,722 basepairs (bp) of DNA 40 kilobases (kb) centromeric of HLA-B. The sequence was obtained from single-stranded (M13) and double-stranded (pUC19) templates of mapped or randomly shot-gun subcloned DNA fragments that were derived from the cosmid M32A (Spies et al., 1989). The first exon encoding the leader peptide is followed by an intron of 6840 bp, which is unusually large for a class I gene. The remainder of the MICA gene shows an organization quite similar to that of conventional class I genes, except for the presence of a relatively long intron following the transmembrane exon and the fusion of the cytoplasmic tail and 3′ untranslated sequence in a single last exon.

The MICB gene has been mapped in cloned cosmids by DNA blot hybridizations using a MICA cDNA probe. It corresponds to mRNA of about 2.4 kb, distinct from MICA mRNA, which is 1.4 kb in size (Bahram et al., 1994). A partial 2304 base pairs (bp) MICB cDNA clone lacking the leader peptide sequence was isolated from an IMR90 human lung fibroblast library by screening with the MICA cDNA probe. The missing 5′ end sequence was cloned by a 5′ Rapid Amplification of cDNA ends polymerase chain reaction (RACE-PCR) procedure after reverse transcription (RT) of poly(A)+HeLa cell mRNA. A cDNA including the complete MICB coding sequence was subsequently generated by RT-PCR and cloned. The full-length MICB cDNA sequence of 2380 bp encodes a polypeptide of 383 amino acids that begins with a probable translation initiation codon (ATG) at nucleotide position 6 (Bahram and Spies, 1996). The stop codon is followed by a relatively long 3′ untranslated region, which accounts for the size difference of the MICB and MICA mRNAs. A consensus polyadenylation signal near the 3′ end of the MICB cDNA is missing; the nearest AATAAA sequence is located 772 bp upstream and an appropriately positioned alternative polyadenylation signal is not readily discernible.

The MICB translation product is identical to the MICA chain in length and domain organization and is highly similar, with 83% matching amino acid residues. Of the total of 65 amino acid substitutions, 18 are clustered within a segment of 24 amino acids in the putative transmembrane segment of MICB, which represents the sole highly disparate portion of the aligned sequences. In the α1-α3 domains, MICB and MICA share 86% amino acid sequence similarity, with 15, 14, and 8 amino acid substitutions in the α1, α2, and α3 domains, respectively, which show no notable preferential distribution. Like MICA, the putative MICB chain may be heavily glycosylated, owing to the presence of five potential N-linked glycosylation sites, of which four in the α3 domain are common to both sequences. None of the three N-linked glycosylation motifs in MICA a1 and α2 are conserved in MICB, which has one such motif in the α2 domain. The highly conserved glycosylation site at amino acid position 86 in MHC class I chains is missing in MICB and MICA. Both sequences include the two pairs of cysteines in the α2 and α3 domains, which form intradomain disulfide bonds in class I chains, and several extra cysteine residues.

Common to MICB and MICA is a gap in the α1 domain, which corresponds to the peptide side chain-binding pocket B (“45” pocket) in many MHC class I chains, and an insertion of 6 amino acids at position 147 in the α2 domain (Bahram et al., 1994). Overall, MICB shows the same degree of divergence from mammalian MHC class I chains as MICA, with most of the amino acid residues that are invariant among vertebrate class I sequences being conserved (Grossberger and Parham, 1992; Bahram et al., 1994). Thus, altogether, MICB and MICA are very closely related and were probably derived by a relatively recent gene duplication.

Additional sequences similar to MICA and MICB (MICA, MICD, and MICE) have been localized in the human MHC near the HLA-E, -A, and -F genes using yeast artificial chromosome (YAC) clones spanning the class I region (Bahram et al., 1994). By partial genomic sequencing of corresponding cosmid DNA, these three sequences were identified as truncated gene fragments. Thus, MICA and MICB are the only functional members in this family of highly diverged MHC class I genes. This is similar to the existence of numerous class I pseudogenes and gene fragments in the human MHC and mouse H2 complex (Stroynowski, 1990).

The inventors have studied the expression of MIC polypeptides using their specific antibodies, transfected mutant cell lines and epithelial tumor cell lines. The results from these and other experiments established that, contrary to MHC class I molecules, MIC are not associated with β2-microglobulin and peptides (Groh et al., 1996; Groh et al, 1998). Both MICA and MICB are highly glycosylated; the deglycosylated polypeptides are of 43 kilodalton (kD). The crystal structure of MICA shows rearranged domain interfaces precluding binding of β2-microglobulin and the absence of a peptide binding groove (Li et al., 1999). The interaction of MICA with NKG2D homodimers has been refined by the complex crystal structure of these molecules (Li et al., 2001).

In accordance with the present invention, MICA/MICB polypeptides or fragments thereof may be the target of therapeutic intervention, for example, by interfering with their interaction with the NKG2D receptor in the context of cancer therapy, or by enhancing this interaction as a way of augmenting the immunosuppressive effect of NKG2D⁺ CD4⁺ T cells in the context of autoimmune disease. In addition, MICA/MICB may be used as part of a screening assay, such as one that targets the interaction between MICA/MICB and ERP5.

B. MIC-Binding Substances

In one embodiment, the present invention contemplates the use of agents that bind to MICA or MICB, thereby preventing its interaction with agents that bind to MIC, preventing MIC function, and preventing action upon MIC by MIC-modifying agents. One such agent is an antibody that binds to MICA or MICB. In particular, binding to various regions on MICA or MICB is contemplated to confer specific effects on the MIC and/or on MIC-interacting proteins. For example, binding of an antibody to MIC α3 domain, which contains the ERP5 cleavage site, would prevent cleavage but not interfere with actions of other MIC domains, such as binding to NKG2D. Alternatively, one may target an inhibitor antibody to other functional regions of MIC, such as those governing NKG2D binding. The antibody may also be bi-specific, binding to MIC and to another agent such as ERP5.

Other inhibitors of MIC may comprise functional fragments of MIC receptors, which include NKG2D and ERP5. Such receptor fragments could comprise polypeptides lacking transmembrane domains, i.e., soluble forms of the receptors.

### IV. PROTEIN DISULFIDE ISOMERASE ERP5 AND ERP5 ANTAGONISTS

A. ERP5

Formation and rearrangement of disulfide bonds during the correct folding of nascent proteins is modulated by a family of enzymes known as thiol isomerases, which include protein disulfide isomerase (PDI), endoplasmic reticulum protein 5 (ERP5), and ERP57. Recent evidence supports an alternative role for this family of proteins on the surface of cells, where they are involved in receptor remodeling and recognition.

In platelets, blocking PDI with inhibitory antibodies inhibits a number of platelet activation pathways, including aggregation, secretion, and fibrinogen binding. Analysis of human platelet membrane fractions identified the presence of the thiol isomerase protein ERP5. Further study showed that ERP5 is resident mainly on platelet intracellular membranes, although it is rapidly recruited to the cell surface in response to a range of platelet agonists. Blocking cell-surface ERP5 using inhibitory antibodies leads to a decrease in platelet aggregation in response to agonists, and a decrease in fibrinogen binding and P-selectin exposure. It is possible that this is based on the disruption of integrin function, as the inventors observed that ERP5 becomes physically associated with the integrin β(3) subunit during platelet stimulation (Jordan et al., 2005).

B. Antagonists

Inhibitors of ERP5 may comprise an inhibitor or ERP5 function or expression. Inhibitors of ERP5 expression include antisense, ribozymes and siRNA molecules that target ERP5 coding sequences and/or transcription/translation signals. Other antagonists include binding agents for ERP5, such as antibodies or fragments of MIC, including the α3 domain of MICA or MICB. Finally, drugs (organopharmaceuticals) may be provided that down-regulate the expression, intracellular processing or cell-surface transport of the ERP5.

### V. FAS LIGAND

Apoptosis occurs both during programmed cell death and as part of a process induced by some cytotoxic T cells. Fas ligand (FasL) was identified as being involved in the signaling that triggers CTL mediated cell death by binding to the cell surface receptor variously known as Fas. FasL is expressed not only in the cells of the immune system, but also in the liver, lung, ovary, and heart—its function in these tissues is unclear. FasL has 281 amino acids and an approximate molecular weight of 32 kDa. The gene for FasL is located on chromosome 1q23. FasL is a type II transmembrane protein that belongs to the tumor necrosis factor family.

FasL is expressed in activated splenocytes and thymocytes, consistent with its involvement in T-cell-mediated death. It is proteolytically cleaved at the cell surface and released into the extracellular fluid. FasL occurs in two alternatively spliced forms. FasL, in conjunction with Fas, plays a pivotal role in T-cell development and clonal deletion of self-reactive T cells. Binding of FasL to its receptor activates death domain proteins, which in turn activate various caspase family members (cysteine-dependent, aspartate-specific proteases that target specific enzymes involved in cell function), leading to cell death.

### VI. ASSAYS

In accordance with the present invention, a variety of assay formats maybe used to screen for the activity, expression, secretion, binding, cleavage, modification, internalization or degradation of various molecules including MICA, MICB, ERP5 and NKG2D. Such assays include immunological assays, such a RIA, ELISA, Western blot, immunoprecipitation, and immunohistochemistry. Other assays include affinity assays, pseudo-affinity assays (including both filter and column formats; competitive and non-competitive), cell sorting assays (FACS), mass spectrometry, scintillation proximity assays, fluorescent quenching assays, as well as others. The steps of various useful immunodetection methods have been described in the scientific literature (e.g., Doolittle and Ben-Zeev, 1999; Gulbis and Galand, 1993; De Jager et al., 1993; Nakamura et al., 1987, each incorporated herein by reference). In addition, assays for nucleic acid expression may be used as a surrogate for protein expression or cellular activation. Some of these are described below.

A. Immunoassays

In a specific embodiment, useful antibodies include CX5 (Ebioscience catalog number 14-5882), 1D11, BAT221, ECM217, and ON72 (see, e.g., Groh et al., 2003; André et al., 2004; the entire disclosures of which are herein incorporated by reference). In general, immunobinding methods include obtaining a sample suspected of containing a relevant polypeptide, and contacting the sample with a first antibody under conditions effective to allow the formation of immunocomplexes. Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to, any antigens present. After this time, the sample-antibody composition, such as a tissue section, ELISA plate, dot blot or western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. U.S. patents concerning the use of such labels include 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody and/or a biotin/avidin ligand binding arrangement, as is known in the art.

The antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding ligand, such as an antibody, that has binding affinity for the antibody is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection designed by Charles Cantor uses two different antibodies. A first step biotinylated, monoclonal or polyclonal antibody is used to detect the target antigen(s), and a second step antibody is then used to detect the biotin attached to the complexed biotin. In that method the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

In one exemplary ELISA, the antibodies of the invention are immobilized onto a selected surface exhibiting protein affinity, such as a well in a polystyrene microtiter plate. Then, a test composition suspected of containing the antigen, such as a clinical sample, is added to the wells. After binding and washing to remove non-specifically bound immune complexes, the bound antigen may be detected. Detection is generally achieved by the addition of another antibody that is linked to a detectable label. This type of ELISA is a simple “sandwich ELISA.” Detection may also be achieved by the addition of a second antibody, followed by the addition of a third antibody that has binding affinity for the second antibody, with the third antibody being linked to a detectable label.

In another exemplary ELISA, the samples suspected of containing the antigen, including whole or fragmented cells, are immobilized onto the well surface and then contacted with the anti-ORF message and anti-ORF translated product antibodies of the invention. After binding and washing to remove non-specifically bound immune complexes, the bound anti-ORF message and anti-ORF translated product antibodies are detected. Where the initial anti-ORF message and anti-ORF translated product antibodies are linked to a detectable label, the immune complexes may be detected directly. Again, the immune complexes may be detected using a second antibody that has binding affinity for the first anti-ORF message and anti-ORF translated product antibody, with the second antibody being linked to a detectable label.

Another ELISA in which the antigens/cells are immobilized, involves the use of antibody competition in the detection. In this ELISA, labeled antibodies against an antigen are added to the wells, allowed to bind, and detected by means of their label. The amount of an antigen in an unknown sample is then determined by mixing the sample with the labeled antibodies against the antigen during incubation with coated wells. The presence of an antigen in the sample acts to reduce the amount of antibody against the antigen available for binding to the well and thus reduces the ultimate signal. This is also appropriate for detecting antibodies against an antigen in an unknown sample, where the unlabeled antibodies bind to the antigen-coated wells and also reduces the amount of antigen available to bind the labeled antibodies.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background. The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C., or may be overnight at about 4° C. or so.

The antibodies of the present invention may also be used in conjunction with both fresh-frozen and/or formalin-fixed, paraffin-embedded tissue blocks prepared for study by immunohistochemistry (IHC). The method of preparing tissue blocks from these particulate specimens has been successfully used in previous IHC studies of various prognostic factors, and/or is well known to those of skill in the art (Brown et al., 1990; Abbondanzo, 1999; Allred et al., 1990a; Allred et al., 1990b).

Also contemplated in the present invention is the use of immunohistochemistry. This approach uses antibodies to detect and quantify antigens in intact tissue samples. Generally, frozen-sections are prepared by rehydrating frozen “pulverized” tissue at room temperature in phosphate buffered saline (PBS) in small plastic capsules; pelleting the particles by centrifugation; resuspending them in a viscous embedding medium (OCT); inverting the capsule and pelleting again by centrifugation; snap-freezing in −70° C. isopentane; cutting the plastic capsule and removing the frozen cylinder of tissue; securing the tissue cylinder on a cryostat microtome chuck; and cutting 25-50 serial sections.

Permanent-sections may be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and/or embedding the block in paraffin; and cutting up to 50 serial permanent sections.

B. Mass Spectrometry

By exploiting the intrinsic properties of mass and charge, mass spectrometry (MS) can resolved and confidently identified a wide variety of complex compounds, including nucleic acids and proteins. Traditional quantitative MS has used electrospray ionization (ESI) followed by tandem MS (MS/MS) (Chen et al., 2000; Zhong et al., 2001; Wu et al., 2000) while newer quantitative methods are being developed using matrix assisted laser desorption/ionization (MALDI) followed by time of flight (TOF) MS (Bucknall et al., 2002; Mirgorodskaya et al., 2000; Gobom et al., 2000).

C. Nucleic Acid Detection

In alternative embodiments for detecting protein expression, one may assay for gene transcription. For example, an indirect method for detecting protein expression is to detect mRNA transcripts from which the proteins are made. These methods fundamentally rely on nucleic acid hybridization. Hybridization is defined as the ability of a nucleic acid to selectively form duplex molecules with complementary stretches of DNAs and/or RNAs. Depending on the application envisioned, one would employ varying conditions of hybridization to achieve varying degrees of selectivity of the probe or primers for the target sequence.

Typically, a probe or primer of between 13 and 100 nucleotides, preferably between 17 and 100 nucleotides in length up to 1-2 kilobases or more in length will allow the formation of a duplex molecule that is both stable and selective. Molecules having complementary sequences over contiguous stretches greater than 20 bases in length are generally preferred, to increase stability and selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

For certain applications, for example, lower stringency conditions may be used. Under these conditions, hybridization may occur even though the sequences of the hybridizing strands are not perfectly complementary, but are mismatched at one or more positions. Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Hybridization conditions can be readily manipulated depending on the desired results.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772. The relevant portions of these and other references identified in this section of the Specification are incorporated herein by reference. Since many mRNAs are present in relatively low abundance, nucleic acid amplification greatly enhances the ability to assess expression. A number of template dependent processes are available to amplify the oligonucleotide sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1988, each of which is incorporated herein by reference in their entirety. A reverse transcriptase PCR™ amplification procedure may be performed to quantify the amount of mRNA amplified. Methods of reverse transcribing RNA into cDNA are well known (see Sambrook et al., 1989). Alternative methods for reverse transcription utilize thermostable DNA polymerases. These methods are described in WO 90/07641. Polymerase chain reaction methodologies are well known in the art. Representative methods of RT-PCR are described in U.S. Pat. No. 5,882,864. Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/US89/01025, each of which is incorporated herein by reference in its entirety.

Following any amplification, it may be desirable to separate the amplification product from the template and/or the excess primer. In one embodiment, amplification products are separated by agarose, agarose-acrylamide or polyacrylamide gel electrophoresis using standard methods (Sambrook et al., 1989). Separated amplification products may be cut out and eluted from the gel for further manipulation. Using low melting point agarose gels, the separated band may be removed by heating the gel, followed by extraction of the nucleic acid. Separation of nucleic acids may also be effected by chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

Other methods of nucleic acid detection that may be used in the practice of the instant invention are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference.

D. T Cell Assays

A variety of different T cells assays may be conducted as part of the present invention. The most common form of assay is the proliferation assay. These assays look at the increase in number of T cells in a population and may use a variety of different techniques. A standard assay is a ³H-thymidine incorporation assay. This assay looks at the inclusion in newly synthesized DNA of a triated nucleic acid—typically thymidine—as a surrogate for DNA replication, cell division, and hence proliferation. Another T cell proliferation assay is a cell sorting assay. The typical form is a fluorescence cell sorting assay, where the cells are fluorescently labeled and sorted (i.e., counted) in an automated fashion.

Other T cell assays look for activity on other cells, such as by signaling molecules. This can be accomplished by providing a test population of cells upon which T cells can act. Then, either by direct contact of the test population, or after physical separation but permitting T cell-produced soluble factors to affect the target population, the response of the test population is monitored. Such response my include proliferation, cell death, secretion of another factor, gene expression, antigen presentation, or other activity.

Other T cell assays are found in U.S. Pat. Nos. 6,040,152, 5,356,779, 5,068,174, 4,845,026 and 4,725,669, each of which are hereby incorporated by reference.

### VII. PURIFICATION METHODS

Within certain embodiments of the present invention, one may wish to purify NKG2D, MICA, MICB, ERP5, fragments thereof, or related polypeptides or peptide products. Protein purification techniques are well known to those of skill in the art. These techniques tend to involve the fractionation of the cellular milieu to separate the peptides or polypeptides from other components of the mixture. Having separated peptides or polypeptides from the other plasma components, the peptide or polypeptide sample may be purified using chromatographic and electrophoretic techniques to achieve complete purification. Analytical methods particularly suited to the preparation of a pure peptide are ion-exchange chromatography, exclusion chromatography; polyacrylamide gel electrophoresis; isolectric focusing. A particularly efficient method of purifying peptides is fast protein liquid chromatography or even HPLC.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulphate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater-fold purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al., 1977).

It will therefore be appreciated that under differing electrophoresis conditions, the apparent molecular weights of purified or partially purified expression products may vary.

High Performance Liquid Chromatography PLC) is characterized by a very rapid separation with extraordinary resolution of peaks. This is achieved by the use of very fine particles and high pressure to maintain an adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (alter pH, ionic strength, temperature, etc.).

A particular type of affinity chromatography useful in the purification of carbohydrate containing compounds is lectin affinity chromatography. Lectins are a class of substances that bind to a variety of polysaccharides and glycoproteins. Lectins are usually coupled to agarose by cyanogen bromide. Conconavalin A coupled to Sepharose was the first material of this sort to be used and has been widely used in the isolation of polysaccharides and glycoproteins other lectins that have been include lentil lectin, wheat germ agglutinin which has been useful in the purification of N-acetyl glucosaminyl residues and Helix pomatia lectin. Lectins themselves are purified using affinity chromatography with carbohydrate ligands. Lactose has been used to purify lectins from castor bean and peanuts; maltose has been useful in extracting lectins from lentils and jack bean; N-acetyl-D galactosamine is used for purifying lectins from soybean; N-acetyl glucosaminyl binds to lectins from wheat germ; D-galactosamine has been used in obtaining lectins from clams and L-fucose will bind to lectins from lotus.

The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand. One of the most common forms of affinity chromatography is immunoaffinity chromatography. The generation of antibodies that would be suitable for use in accord with the present invention is discussed elsewhere in the specification.

### VIII. SCREENING METHODS

A variety of different methods may be employed to screen for MIC-related, NKG2D-related and ERP5-related interactions. Any of a large number of assays, both molecular, cell-based, and animal-based models can be used. In typical embodiments, cell-based assays will be used in which cells expressing various targets are exposed to candidate substances and the effect of that candidate substance is assessed.

Any suitable physiological change that reflects NKG2D activity, particularly on CD4⁺ cells, can be used to assess the utility of a test compound or antibody. For example, one can measure a variety of effects, such as changes in gene expression, cell growth, cell proliferation, pH, intracellular second messengers, e.g., Ca²⁺, IP3, cGMP, or cAMP, or activity such as the ability to activate or inhibit other T cells, in particular, CD4⁺ NKG2D⁻ cells. In one embodiment, the activity of the receptor is assessed by detecting the expression of NKG2D-responsive genes, e.g., CD25, IFN-γ, or TNF-α (see, e.g., Groh et al., 2003; André et al., 2004). In one embodiment, NKG2D activity is assessed by incubating CD4⁺ NKG2D⁺ cells in the presence of a ligand (e.g., MICA/MICB) or activating anti-NKG2D antibody, optionally with an anti-CD3 antibody, and assessing the ability of the compound or test antibody to inhibit the release of TNF-α or IFN-γ by the T cells. In another embodiment, CD4⁺ NKG2D⁺ T cells are incubated in the presence of ligand, e.g., MICA, MICB, ULBP-1, ULBP-2, ULBP-3, etc., or ligand-producing cells, e.g., MIC-producing tumor cells, and the ability of the test compound to alter cytokine production (e.g., IFN-γ or TNF-α), FasL secretion, cell growth or proliferation, T cell help, or T cell immunosuppression, is assessed.

In a distinct embodiment, one may examine the interaction between MICA/MICB and the disulphide isomerase ERP5. This interaction may involve a cell free system, i.e., a simple binding assay (filter-based, affinity column, gel exclusion, gel-shift assay), but it may also advantageously incorporate a cell-based system with one or both of MICA/MICB and ERP5 being expressed (for example, on the cell surface) by the cell. The cell may naturally express these molecules, or may have been engineered to express or overexpress the molecules. In addition, MIC or ERP5 may be a variants that includes moieties that facilitate identification, such as epitopes that are recognized by antibodies, 6x-His tags. Alternatively, the MIC or ERP5 may include a label (fluorescent, chemiluminescent, dyes, enzymes). The assay may, as an alternative to looking at binding, look at the effects that ERP5 has on MICA/MICB, such as tetramer formation, disulphide bond formation/dissolution, as well as the effects by other proteins that are facilitated by ERP5, such as MICA/MICB cleavage. One may also look directly at effects on ERP5, such as mRNA or protein expression, maturation, cellular trafficking, cell surface expression and/or stability.

A useful tool for examining gene expression, as discussed above, is a nucleic acid array. Such arrays are commercially available from a variety of sources (e.g., Affymetrix) and involve a plurality of nucleic acid sequences fixed in a particular fashion on the surface of a support such as a chip or wafer. The plurality of nucleic acids sequences represents a plurality of different target genes, and hybridization of mRNA from a target cell to the array, followed by detection, can demonstrate expression of a target gene as well as relative amounts.

In animal-based assays, any physiological or pathological consequence of NKG2D modulation in CD4⁺ cells within the animal can be used to assess antibody or test compound activity. For example, in one embodiment, CD4⁺ NKG2D⁺ cells are introduced into the joints of an animal model, with or without co-administration of ligand producing cells such as MICA-producing synoviocytes, and inflammation or tissue damage is assessed. Test compounds or antibodies can then be introduced, and their ability to inhibit, slow, reverse, or in any way affect the inflammation or tissue damage is detected. Another embodiment involves the use of CD4⁺ NKG2D⁺ cells in an animal afflicted with a tumor that produces MICA/MICB, and that also contains CD4⁺ NKG2D⁻ cells. Animal based assays may also be used to examine the MICA/MICB-ERP5 interaction, particularly in the context of an animal bearing a tumor that expresses MICA/MCA-ERP5.

A. Candidate Substances

The candidate compounds can be any small molecule compound, or a biological entity, such as a protein, sugar, nucleic acid or lipid. Typically, test compounds will be small chemical molecules and peptides. Essentially, any chemical compound can be used as a potential NKG2D, MIC or ERP5 agonist or antagonist, although most often compounds that can be dissolved in aqueous or organic (especially DMSO-based) solutions are used. In general, assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs, Switzerland) and the like.

Preparation and screening of combinatorial chemical libraries is well known to those of skill in the art. Such combinatorial chemical libraries include, but are not limited to, peptide libraries (see, e.g., U.S. Pat. No. 5,010,175, Furka, 1991, and Houghton et al., 1991). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., 1993), vinylogous polypeptides (agihara et al., 1992), nonpeptidal peptidomimetics with glucose scaffolding (Hirschmann et al., 1992), analogous organic syntheses of small compound libraries (Chen et al., 1994), oligocarbamates (Cho et al.), and/or peptidyl phosphonates (Campbell et al., 1994), nucleic acid libraries (see Ausubel et al., 1996; Sambrook et al., 1989), peptide nucleic acid libraries (see, e.g. U.S. Pat. No. 5,539,083), antibody libraries (see, e.g., Vaughn et al., 1996, and PCT/US96/10287), carbohydrate libraries (see, e.g., Liang et al., 1996, and U.S. Pat. No. 5,593,853), small organic molecule libraries (see, e.g., benzodiazepines, Baum, 1993; isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 NPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Tripos, Inc., St. Louis, Mo., 3D Pharmaceuticals, Exton, Pa., Martek Biosciences, Columbia, Md., etc.).

Using lead compounds to help develop improved compounds is know as “rational drug design” and includes not only comparisons with know inhibitors and activators, but predictions relating to the structure of target molecules. The goal of rational drug design is to produce structural analogs of biologically active polypeptides or target compounds. By creating such analogs, it is possible to fashion drugs, which are more active or stable than the natural molecules, which have different susceptibility to alteration or which may affect the function of various other molecules. In one approach, one would generate a three-dimensional structure for a target molecule, or a fragment thereof. This could be accomplished by x-ray crystallography, computer modeling or by a combination of both approaches. It also is possible to use antibodies to ascertain the structure of a target compound activator or inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

Candidate compounds may include fragments or parts of naturally-occurring compounds, or may be found as active combinations of known compounds, which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be peptide, polypeptide, polynucleotide, small molecule inhibitors or any other compounds that may be designed through rational drug design starting from known inhibitors or stimulators.

B. Assay Formats

To identify a modulator, one generally will determine the function of a target in the presence and absence of the candidate substance, a modulator defined as any substance that alters function of a target. For example, a method generally comprises:
(a) providing a candidate modulator;
(b) admixing the candidate modulator with a target compound, cell, or experimental animal;
(c) measuring one or more effects of the candidate on the compound, cell or animal in step (b); and
(d) comparing the effect(s) measured in step (c) with the effect(s) of the candidate on the compound, cell or animal in the absence of the candidate,
wherein a difference between the measured effects in (c) and (d) indicates that the candidate is, indeed, a modulator of the target compound, cell or animal.

In any of the herein-described assays, a modulation of 5%, 10%, 20%, 30%, 40%, 50%, 60%, 70%, 80%, 90%, 95%, or greater modulation in any detectable characteristic, as described above, is contemplated.

It will, of course, be understood that all the screening methods of the present invention are useful in themselves notwithstanding the fact that effective candidates may not be found. The invention provides methods for screening for such candidates, not solely methods of finding them.

C. In vitro Assays

A quick, inexpensive and easy assay to run is an in vitro assay. Such assays generally use isolated molecules, can be run quickly and in large numbers, thereby increasing the amount of information obtainable in a short period of time. A variety of vessels may be used to run the assays, including test tubes, filters, plates, chambers, dishes and other surfaces such as dipsticks or beads.

One example of a cell free assay is a binding assay. While not directly addressing function, the ability of a modulator to bind to a target molecule in a specific fashion is strong evidence of a related biological effect. For example, binding of a molecule to a target may, in and of itself, be inhibitory, due to steric, allosteric or charge-charge interactions. The target may be either free in solution, fixed to a support, such as a filter or column, or expressed in or on the surface of a cell. Either the target or the compound may be labeled, thereby permitting determining of binding. Usually, the target will be the labeled species, decreasing the chance that the labeling will interfere with or enhance binding. Competitive binding formats can be performed in which one of the agents is labeled, and one may measure the amount of free label versus bound label to determine the effect on binding. Such formats could be advantageously applied to the examination of MICA/MICB-ERP5 interactions.

A particular assay format will involve segregating various cell populations from direct contact but that permit soluble cell signaling, as in chambers with subcellular-sized apertures, or with subcellular-permeable capacity, such as gels, agars or resins.

A technique for high throughput screening of compounds is described in WO 84/03564. Large numbers of small peptide test compounds are synthesized on a solid substrate, such as plastic pins or some other surface. Bound polypeptide is detected by various methods.

D. In cyto Assays

The present invention also contemplates the screening of compounds for their ability to modulate actions in whole, living cells, in particular T cells. Various cell lines can be utilized for such screening assays, including cells specifically engineered for this purpose. Alternatively, freshly isolated “primary” cells, such as those obtained from tissue or tumor biopsies, can also be used. Depending on the assay, culture may be required. The cell may be examined using any of a number of different physiologic assays, including protein binding, protein secretion, or effects on other cells or cell types (proliferation, apoptosis, growth). Alternatively, molecular analysis may be performed, for example, looking at protein expression or secretion, mRNA expression (including differential display of whole cell or polyA RNA). A particular assay of this format involves using of CD4⁺ NKG2D⁺ or a supernatant from a culture of these cells for culturing with CD4⁺ NKG2D⁻. The effects on the NKG2D⁻ are then assessed. A related assay would examine the production, by CD4⁺ NKG2D⁺, of FasL in response to treatment with the candidate substance, at is has been determined that the negative immunomodulation of CD4⁺ NKG2D⁺ is effected by FasL. Another assay would examine the effects of a modulator on the MIC-ERP5 interaction in a cell expressing both of these molecules, for example, by looking at MIC disulfide bonding, cleavage or release. Other T cell assay are described above.

E. In vivo Assays

In vivo assays involve the use of various animal models, including transgenic animals that have been engineered to carry markers that can be used to measure the effects in response to treatment with a candidate substance, or to be predisposed to a certain pathology, such as cancer or autoimmune disease. Due to their size, ease of handling, and information on their physiology and genetic make-up, mice are a preferred embodiment, especially for transgenics. However, other animals are suitable as well, including rats, rabbits, hamsters, guinea pigs, gerbils, woodchucks, cats, dogs, sheep, goats, pigs, cows, horses and monkeys (including chimps, gibbons and baboons). Assays for modulators may be conducted using an animal model derived from any of these species.

In such assays, one or more candidate substances are administered to an animal, and the ability of the candidate substance(s) to alter one or more relevant characteristics, as compared to a similar animal not treated with the candidate substance(s), identifies the candidate a modulator. The characteristics may be any of those discussed above, such as ERP5 expression or function, presence of CD4⁺ NKG2D⁺ cells, MICA/MICA cleavage and/or secretion, etc. In these embodiments, the method generally includes the steps of administering a candidate substance to the animal, and determining the ability of the candidate substance to modulate one or more of the foregoing characteristics.

Treatment of these animals with test compounds will involve the administration of the compound, in an appropriate form, to the animal. Administration will be by any route that could be utilized for clinical or non-clinical purposes, including but not limited to oral, nasal, buccal, or even topical. Alternatively, administration may be by intratracheal instillation, bronchial instillation, intradermal, subcutaneous, intramuscular, intraperitoneal or intravenous injection. Specifically contemplated routes are systemic intravenous injection, regional administration via blood or lymph supply, or directly to an affected site.

Determining the effectiveness of a compound in vivo may involve a variety of different criteria, as discussed above. Also, measuring toxicity and dose response can be performed in animals in a more meaningful fashion than in in vitro or in cyto assays.

### IX. THERAPIES

A. Cancer

Epithelial tumors are those that arise from surface or lining tissues. Epithelial cells cover surfaces and line internal passage ways. As such, epithelial tissue is found in 3 major places: outer surfaces of the body; surfaces of organs and internal surface lining of tubules, vessels and hollow organs. Most glands are composed primarily of epithelial cells. Therefore, epithelial tumors may be found on any surface or lining of the body that fits the above description. An “epithelial cancer” is one that is derived from an epithelial tissue and may occur in any location of the body, including ovarian cancer, squamous cell carcinoma, thyroid cancer, mammary neoplasia, and basal cell carcinoma

The inventors have determined that a particular subset of CD4⁺ cells—those expressing NKG2D receptors—are enhanced in cancer patients where the cancer cells produce MICA/MICB. Surprisingly, these cells interfere with the normal helper functions associated with the remaining NKG2D-negative CD4⁺ cells, and thus exhibit a profound negative immunodulatory effect. Furthermore, it has been determined by the inventors that this effect is the result of a soluble factor produced by the NKG2D-positive CD4⁺ cells, namely, FasL.

Thus, the present invention contemplates the treatment of subjects with MICA/MICB producing cancers with agents that inhibit NKG2D⁺ CD4⁺ cells and FasL. Specifically contemplated are agents that bind to NKG2D⁺ CD4⁺ cells and, in so doing, inhibit or eliminate these cells, for example, an anti-NKG2D antibody (including chimeric and humanized forms thereof), optionally linked to a toxin, or MICA/MICB linked to a toxin. Alternatively, the agent may specifically interfere with FasL synthesis or release, or FasL binding to receptors on NKG2D⁻ CD4⁺ cell, such as anti-FasL antibodies, FasL siRNA or FasL antisense molecules. Also contemplated are combination therapies where the foregoing anti-NKG2D or anti-FasL therapy is combined with traditional therapies including radiation, chemotherapy, hormonal therapy and surgery.

In another embodiment, the inhibitory actions of the NKG2D⁺ CD4⁺ cells may be modulated by affecting MICA/MICB cleavage by contacting the cell with a modulator of ERP5 expression or function. The modulator may alter ERP5 release of MICA/MICB, ERP5 binding to MICA/MICB, ERP5 isomerization of MICA/MICB, ERP5 thioreduction of MICA/MICB, or ERP5 transcription or translation or cell surface expression. More specifically, the modulator may be an antagonist that is a competing substrate for ERP5, an antagonist that is a thioreductase inhibitor, or particularly, bacitracin, DTNB or PAO. In particular, the invention encompasses the use of a MICA/MICB-binding agent, such as an antibody. The antibody may, in particular, be selected on the basis of its ability to alter ERP5's thioreduction and/or cleavage of MICA/MICB. The antibody may bind to the α3 domain of MICA/MICB.

Tumor cell resistance to DNA damaging agents represents a major problem in clinical oncology. One goal of current cancer research is to find ways to improve the efficacy of chemo- and radiotherapy. To kill cells, inhibit cell growth, inhibit metastasis, inhibit angiogenesis or otherwise reverse or reduce the malignant phenotype of tumor cells, using the methods and compositions of the present invention, one would generally contact a cancer cell with one of the modulators described herein and at least one other agent. These compositions would be provided in a combined amount effective to kill or inhibit proliferation of the cell. This process may involve contacting the cells with the expression construct and the agent(s) or factor(s) at the same time. This may be achieved by contacting the cell with a single composition or pharmacological formulation that includes both agents, or by contacting the cell with two distinct compositions or formulations, at the same time, wherein one composition includes one agent and the other includes the other agent.

Alternatively, one therapy may precede or follow the other by intervals ranging from minutes to weeks. In embodiments where the agents are applied separately to the cell, one would generally ensure that a significant period of time did not expire between the time of each delivery, such that the agents would still be able to exert an advantageously combined effect on the cell. In such instances, it is contemplated that one would contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either agent will be desired. Various combinations may be employed, where the modulator described above “A” and the other cancer therapy is “B”, as exemplified below:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are contemplated. Again, to achieve cell killing, both agents/treatments are delivered to a cancer cell in a combined amount effective to treat the cell.

Agents or factors suitable for use in a combined therapy are any chemical compound or treatment method that induces DNA damage when applied to a cell. Such agents and factors include radiation and waves that induce DNA damage such as, γ-irradiation, X-rays, UV-irradiation, microwaves, electronic emissions, and the like. A variety of chemical compounds, also described as “chemotherapeutic agents,” function to induce DNA damage, all of which are intended to be of use in the combined treatment methods disclosed herein. Chemotherapeutic agents contemplated to be of use, include, e.g., adriamycin, 5-fluorouracil (5FU), etoposide (VP-16), camptothecin, actinomycin-D, mitomycin C, cisplatin (CDDP) and even hydrogen peroxide. The invention also encompasses the use of a combination of one or more DNA damaging agents, whether radiation-based or actual compounds, such as the use of X-rays with cisplatin or the use of cisplatin with etoposide. In certain embodiments, the use of cisplatin in combination with a Killin expression construct is particularly preferred as this compound.

In treating cancer according to the invention, one would contact the tumor cells with an agent in addition to the modulator of the present invention. This may be achieved by irradiating the localized tumor site with radiation such as X-rays, UV-light, 7-rays or even microwaves. Alternatively, the tumor cells may be contacted with the agent by administering to the subject a therapeutically effective amount of a pharmaceutical composition comprising a compound such as, adriamycin, 5-fluorouracil, etoposide, camptothecin, actinomycin-D, mitomycin C, or more preferably, cisplatin.

Agents that directly cross-link nucleic acids, specifically DNA, are envisaged to facilitate DNA damage leading to a synergistic, antineoplastic combination with Killin. Agents such as cisplatin, and other DNA alkylating agents may be used. Cisplatin has been widely used to treat cancer, with efficacious doses used in clinical applications of 20 mg/m² for 5 days every three weeks for a total of three courses. Cisplatin is not absorbed orally and must therefore be delivered via injection intravenously, subcutaneously, intratumorally or intraperitoneally.

Agents that damage DNA also include compounds that interfere with DNA replication, mitosis and chromosomal segregation. Such chemotherapeutic compounds include adriamycin, also known as doxorubicin, etoposide, verapamil, podophyllotoxin, and the like. Widely used in a clinical setting for the treatment of neoplasms, these compounds are administered through bolus injections intravenously at doses ranging from 25-75 mg/m² at 21 day intervals for adriamycin, to 35-50 mg/m² for etoposide intravenously or double the intravenous dose orally.

Agents that disrupt the synthesis and fidelity of nucleic acid precursors and subunits also lead to DNA damage. As such a number of nucleic acid precursors have been developed. Particularly useful are agents that have undergone extensive testing and are readily available. As such, agents such as 5-fluorouracil (5-FU), are preferentially used by neoplastic tissue, making this agent particularly useful for targeting to neoplastic cells. Although quite toxic, 5-FU, is applicable in a wide range of carriers, including topical, however intravenous administration with doses ranging from 3 to 15 mg/kg/day being commonly used.

Other factors that cause DNA damage and have been used extensively include what are commonly known as 7-rays, X-rays, and/or the directed delivery of radioisotopes to tumor cells. Other forms of DNA damaging factors are also contemplated such as microwaves and UV-irradiation. It is most likely that all of these factors effect a broad range of damage DNA, on the precursors of DNA, the replication and repair of DNA, and the assembly and maintenance of chromosomes. Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

The skilled artisan is directed to “Remington's Pharmaceutical Sciences” 15th Edition, chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

The inventors propose that the local or regional delivery of modulators of the present invention to patients with cancer will be a very efficient method for treating the clinical disease. Similarly, the chemo- or radiotherapy may be directed to a particular, affected region of the subjects body. Alternatively, systemic delivery of either agent may be appropriate in certain circumstances, for example, where extensive metastasis has occurred.

In addition, it will be useful to screen for MICA/MICB-expressing cancers. This information is contained in previously published documents (PCT/US03/12299), which are hereby incorporated by reference. It is contemplated that both surface bound and soluble MIC may be detected by assaying for MIC polypeptides, including MICA (2C10 and 3H5) and MICA & MICB (6D4 and 6G6).

B. Autoimmune Disease

An autoimmune disease or condition is characterized by an underlying defect in which there is an immune response against the body's own organs and/or tissues. There are believed to be at least 80 such conditions and diseases, which include, but are not limited to, the following: Alopecia Areata, Ankylosing Spondylitis, Antiphospholipid Syndrome, Autoimmune Addison's Disease, Autoimmune Hemolytic Anemia, Autoimmune Hepatitis, Behcet's Disease, Bullous Pemphigoid, Cardiomyopathy, Celiac Sprue-Dermatitis, Chronic Fatigue Immune Dysfunction Syndrome (CFIDS), Chronic Inflammatory Demyelinating Polyneuropathy, Churg-Strauss Syndrome, Cicatricial Pemphigoid, CREST Syndrome, Cold Agglutinin Disease, Crohn's Disease, Discoid Lupus, Essential Mixed Cryoglobulinemia, Fibromyalgia-Fibromyositis, Graves' Disease, Guillain-Barré, Hashimoto's Thyroiditis, Idiopathic Pulmonary Fibrosis, Idiopathic Thrombocytopenia Purpura (ITP), IgA Nephropathy, Insulin-dependent Diabetes, Juvenile Arthritis, Lichen Planus, Meniere's Disease, Mixed Connective Tissue Disease, Multiple Sclerosis, Myasthenia Gravis, Pemphigus Vulgaris, Pernicious Anemia, Polyarteritis Nodosa, Polychondritis, Polyglandular Syndromes, Polymyalgia Rheumatica, Polymyositis and Dermatomyositis, Primary Agammaglobulinemia, Primary Biliary Cirrhosis, Psoriasis, Raynaud's Phenomenon, Reiter's Syndrome, Rheumatic Fever, Rheumatoid Arthritis (RA), Sarcoidosis, Scleroderma, Sjogren's Syndrome, Stiff-Man Syndrome, Systemic Lupus Erythematosus (SLE), pediatric SLE, Takayasu Arteritis, Temporal Arteritis/Giant Cell Arteritis, Ulcerative Colitis, Uveitis, Vasculitis, Vitiligo, and Wegener's Granulomatosis. Also contemplated are transplant scenarios (bone marrow transplant, solid organ allografting), where T-cell mediated responses give rise to, e.g., graft-versus-host disease. Methods and compositions of the invention are specifically contemplated for use with respect to RA.

Though previous work by the inventors suggests that NKG2D-positive CD4⁺ CD28⁻ T cells are causative in autoimmune disorders, the identification of an immunosuppressive function in this cell population raises the possibility that it can be exploited in the treatment of autoimmunity. In this context, one would provide a subject suffering from the autoimmune disease with an agonist of NKG2D⁺ CD4⁺ cells, or a FasL agonist (or FasL itself for that matter).

In another embodiment, the actions of the NKG2D⁺ CD4⁺ cells in the context of autoimmunity may be modulated by affecting MICA/MICB cleavage by contacting the cell with a modulator of ERP5 expression or function. The modulator may alter ERP5 release of MICA/MICB, ERP5 binding to MICA/MICB, ERP5 isomerization of MICA/MICB, ERP5 thioreduction of MICA/MICB, or ERP5 transcription or translation or cell surface expression.

The treatment may involve multiple rounds of the therapeutic agent. For example, following an initial round of administration, the level and/or activity of NKG2D-expressing, CD4⁺ T cells or FasL signaling in a patient may be re-assessed, and, if appropriate, an additional round of administration can be performed. In this way, multiple rounds of administration can be performed until the disorder is adequately treated.

Combination therapies with additional agents also are contemplated. Corticosteroid drugs, analgesics, non-steroidal anti-inflammatory drugs (NSAIDs) or more powerful immunosuppressant drugs such as cyclophosphamide, methotrexate and azathioprine that suppress the immune response and stop the progression of autoimmune diseases. Radiation of the lymph nodes and plasmapheresis (a procedure that removes the diseased cells and harmful molecules from the blood circulation) are sometimes employed.

In addition, it will be useful to screen for MICA/MICB-expressing cells in autoimmune patients. This information is contained in previously published documents (PCT/US03/12299), which are hereby incorporated by reference. It is contemplated that both surface bound and soluble MIC may be detected by assaying for MIC polypeptides, including MICA (2C10 and 3H5) and MICA & MICB (6D4 and 6G6).

C. Inflammatory Diseases

The present invention also permits the treatment of various inflammatory diseases, where the inflammation is mediated by CD4⁺ CD28⁻ T cells that express NKG2D, which are stimulated by the stress-inducible MIC ligands. So of these disease states are discussed below.

Psoratic Arthritis. Psoriasis is an inflammatory and proliferative skin disorder with a prevalence of 1.5-3%. Approximately 20% of patients with psoriasis develop a characteristic form of arthritis that has several patterns (Gladman, 1992; Moll & Wright, 1973; Jones et al., 1994; Gladman et al., 1995). Some individuals present with joint symptoms first but in the majority, skin psoriasis presents first. About one-third of patients have simultaneous exacerbations of their skin and joint disease (Gladman et al., 1987) and there is a topographic relationship between nail and distal interphalangeal joint disease (Jones et al., 1994; 33:834-9; V. Wright, 1956). Although the inflammatory processes which link skin, nail and joint disease remain elusive, an immune-mediated pathology is implicated.

Psoriatic arthritis (PsA) is a chronic inflammatory arthropathy characterized by the association of arthritis and psoriasis and was recognized as a clinical entity distinct from rheumatoid arthritis (RA) in 1964 (Blumberg et al., 1964). Subsequent studies have revealed that PsA shares a number of genetic, pathogenic and clinical features with other spondyloarthropathies (SpAs), a group of diseases that comprise ankylosing spondylitis, reactive arthritis and enteropathic arthritis (Wright, 1979). The notion that PsA belongs to the SpA group has recently gained further support from imaging studies demonstrating widespread enthesitis in the, including PsA but not RA (McGonagle et al., 1999; McGonagle et al., 1998). More specifically, enthesitis has been postulated to be one of the earliest events occurring in the SpAs, leading to bone remodeling and ankylosis in the spine, as well as to articular synovitis when the inflamed entheses are close to peripheral joints. However, the link between enthesitis and the clinical manifestations in PsA remains largely unclear, as PsA can present with fairly heterogeneous patterns of joint involvement with variable degrees of severity (Marsal et al., 1999; Salvarani et al., 1998). Thus, other factors must be posited to account for the multifarious features of PsA, only a few of which (such as the expression of the HLA-B27 molecule, which is strongly associated with axial disease) have been identified. As a consequence, it remains difficult to map the disease manifestations to specific pathogenic mechanisms, which means that the treatment of this condition remains largely empirical.

Family studies have suggested a genetic contribution to the development of PsA (Moll & Wright, 1973). Other chronic inflammatory forms of arthritis, such as ankylosing spondylitis and rheumatoid arthritis, are thought to have a complex genetic basis. However, the genetic component of PsA has been difficult to assess for several reasons. There is strong evidence for a genetic predisposition to psoriasis alone that may mask the genetic factors that are important for the development of PsA. Although most would accept PsA as a distinct disease entity, at times there is a phenotypic overlap with rheumatoid arthritis and ankylosing spondylitis. Also, PsA itself is not a homogeneous condition and various subgroups have been proposed. Although not all these confounding factors were overcome in the present study, we concentrated on investigating candidate genes in three broad categories of patients with PsA that cover the disease spectrum.

Polymorphisms in the promoter region of the TNFA region are of considerable interest as they may influence levels of TNF-α secretion (Jacob et al., 1990; Bendzen et al., 1988). Increased amounts of TNF-α have been reported in both psoriatic skin (Ettehadi et al., 1994) and synovial fluid (Partsch et al., 1997).

Recent trials have shown a positive benefit of anti-TNF treatment in both PsA (Mease et al., 2000) and ankylosing spondylitis (Brandt et al., 2000). Furthermore, the locus for TNF-α resides within the class III region of the MHC and thus may provide tighter associations with PsA than those provided by flanking class I and class II regions. There were relatively weak associations with the TNFA alleles in our total PsA group. The uncommon TNFA-238A allele was increased in frequency in the group with peripheral polyarthritis and absent in those patients with spondylitis, although this finding may be explained by linkage disequilibrium with HLA-Cw*0602. Whether there are functional consequences associated with polymorphisms at the TNFA-238 allele is unclear (Pociot et al., 1995). Nonetheless, it is possible that the pattern of arthritis that develops in patients with psoriasis may be linked directly or indirectly to this particular allele.

Hohler et al. (1997), (A TNF-α promoter polymorphism is associated with juvenile onset psoriasis and psoriatic arthritis) found an increase in the frequency of the TNFA-238A allele in patients with PsA as well as in juvenile onset psoriasis. The association of TNFA-238A with both juvenile onset psoriasis and PsA was stronger than that with HLA-Cw6. Similarly, in our study, there were strong associations between juvenile onset psoriasis and both HLA-Cw*0602 and TNFA-238A, although neither allele had any relationship to the age of onset of arthritis. In our study, all patients with PsA who had at least one TNFA-238A allele were HLA-Cw6-positive, emphasizing the close linkage between these alleles in PsA. However, in contrast to the study by Hohler et al. (1997), and explainable by close linkage to HLA-CW*0602, the TNFA-238A allele was only increased in patients with peripheral arthritis. It is also of interest that, in a separate study of ankylosing spondylitis, the same group found the uncommon TNFA-308A and -238A alleles to have a protective effect on the development of spondylitis (Hohler et al., 1998).

Reactive Arthritis. In reactive arthritis (ReA) the mechanism of joint damage is unclear, but it is likely that cytokines play critical roles. A more prevalent Th1 profile high levels of interferon gamma (IFN-γ) and low levels of interleukin 4 (L-4) has been reported (Lahesmaa et al., 1992; Schlaak et al., 1992; Simon et al., 1993; Schlaak et al., 1996; Kotake et al., 1999; Ribbens et al., 2000), but several studies have shown relative predominance of IL-4 and IL-10 and relative lack of IFN-γ and tumour necrosis factor alpha (TNF-α) in the synovial membrane (Simon et al., 1994; Yin et al., 1999) and fluid (SF) (Yin et al., 1999; Yin et al., 1997) of reactive arthritis patients compared with rheumatoid arthritis (RA) patients. A lower level of TNF-α secretion in reactive arthritis than in RA patients has also been reported after ex vivo stimulation of peripheral blood mononuclear cells PBMC) (Braun et al., 1999).

It has been argued that clearance of reactive arthritis-associated bacteria requires the production of appropriate levels of IFN-γ and TNF-α, while IL-10 acts by suppressing these responses (Autenrieth et al., 1994; Sieper & Braun, 1995). IL-10 is a regulatory cytokine that inhibits the synthesis of IL-12 and TNF-γ by activated macrophages (de Waal et al., 1991; Hart et al., 1995; Chomarat et al., 1995) and of IFN-γ by T cells (Macatonia et al., 1993).

Enteropathic Arthritis. Enteropathic arthritis (EA) occurs in combination with inflammatory bowel diseases (IBD) such as Crohn's disease or ulcerative colitis. It also can affect the spine and sacroiliac joints. Enteropathic arthritis involves the peripheral joints, usually in the lower extremities such as the knees or ankles. It commonly involves only a few or a limited number of joints and may closely follow the bowel condition. This occurs in approximately 11% of patients with ulcerative colitis and 21% of those with Crohn's disease. The synovitis is generally self-limited and non-deforming.

Enteropathic arthropathies comprise a collection of rheumatologic conditions that share a link to GI pathology. These conditions include reactive (i.e., infection-related) arthritis due to bacteria (e.g., Shigella, Salmonella, Campylobacter, Yersinia species, Clostridium difficile), parasites (e.g., Strongyloides stercoralis, Taenia saginata, Giardia lamblia, Ascaris lumbricoides, Cryptosporidium species), and spondyloarthropathies associated with inflammatory bowel disease (IBD). Other conditions and disorders include intestinal bypass (jejunoileal), arthritis, celiac disease, Whipple disease, and collagenous colitis.

The precise causes of enteropathic arthropathies are unknown. Inflammation of the GI tract may increase permeability, resulting in absorption of antigenic material, including bacterial antigens. These arthrogenic antigens may then localize in musculoskeletal tissues (including entheses and synovium), thus eliciting an inflammatory response. Alternatively, an autoimmune response may be induced through molecular mimicry, in which the host's immune response to these antigens cross-reacts with self-antigens in synovium.

Of particular interest is the strong association between reactive arthritis and HLA-B27, an HLA class I molecule. A potentially arthrogenic, bacterially derived antigen peptide could fit in the antigen-presenting groove of the B27 molecule, resulting in a CD8+ T-cell response. HLA-B27 transgenic rats develop features of enteropathic arthropathy with arthritis and gut inflammation.

Familial Mediterranean Fever. Familial Mediterranean Fever is an inherited disorder usually characterized by recurrent episodes of fever and peritonitis (inflammation of the abdominal membrane). In 1997, researchers identified the gene for FMF and found several different gene mutations that cause this inherited rheumatic disease. The gene, found on chromosome 16, codes for a protein that is found almost exclusively in granulocytes—white blood cells important in the immune response. The protein is likely to normally assist in keeping inflammation under control by deactivating the immune response—without this ‘brake,’ an inappropriate full-blown inflammatory reaction occurs: an attack of FMF. To explore whether a molecular diagnostic cytokine characteristic exists, serum samples from six patients with clinically diagnosed FMF were examined and the concentration of cytokines were quantified.

Irritable Bowel Syndrome. Irritable bowel syndrome (IBS) is a functional disorder characterized by abdominal pain and altered bowel habits. This syndrome may begin in young adulthood and can be associated with significant disability. This syndrome is not a homogeneous disorder. Rather, subtypes of IBS have been described on the basis of the predominant symptom—diarrhea, constipation, or pain. In the absence of “alarm” symptoms, such as fever, weight loss, and gastrointestinal bleeding, a limited workup is needed. Once a diagnosis of IBS is made, an integrated treatment approach can effectively reduce the severity of symptoms. IBS is a common disorder, although its prevalence rates have varied. In general, IBS affects about 15% of US adults and occurs about three times more often in women than in men (Jailwala et al., 2000).

IBS accounts for between 2.4 million and 3.5 million visits to physicians each year. It not only is the most common condition seen by gastroenterologists but also is one of the most common gastrointestinal conditions seen by primary care physicians (Everhart et al., 1991; Sandler, 1990).

IBS is also a costly disorder. Compared with persons who do not have bowel symptoms, persons with IBS miss three times as many workdays and are more likely to report being too sick to work (Drossman et al., 1993; Drossman et al., 1997). Moreover, those with IBS incur hundreds of dollars more in medical charges than persons without bowel disorders (Talley et al., 1995). No specific abnormality accounts for the exacerbations and remissions of abdominal pain and altered bowel habits experienced by patients with IBS. The evolving theory of IBS suggests dysregulation at multiple levels of the brain-gut axis. Dysmotility, visceral hypersensitivity, abnormal modulation of the central nervous system (CNS), and infection have all been implicated. In addition, psychosocial factors play an important modifying role. Abnormal intestinal motility has long been considered a factor in the pathogenesis of IBS. Transit time through the small intestine after a meal has been shown to be shorter in patients with diarrhea-predominant IBS than in patients who have the constipation-predominant or pain-predominant subtype (Cann et al., 1983).

In studies of the small intestine during fasting, the presence of both discrete, clustered contractions and prolonged, propagated contractions has been reported in patients with IBS (Kellow & Phillips, 1987). They also experience pain with irregular contractions more often than healthy persons (Kellow & Phillips, 1987; Horwitz & Fisher, 2001)

These motility findings do not account for the entire symptom complex in patients with IBS; in fact, most of these patients do not have demonstrable abnormalities (Rothstein, 2000). Patients with IBS have increased sensitivity to visceral pain. Studies involving balloon distention of the rectosigmoid colon have shown that patients with IBS experience pain and bloating at pressures and volumes much lower than control subjects (Whitehead et al., 1990). These patients maintain normal perception of somatic stimuli.

Multiple theories have been proposed to explain this phenomenon. For example, receptors in the viscera may have increased sensitivity in response to distention or intraluminal contents. Neurons in the dorsal horn of the spinal cord may have increased excitability. In addition, alteration in CNS processing of sensations may be involved (Drossman et al., 1997). Functional magnetic resonance imaging studies have recently shown that compared with control subjects, patients with IBS have increased activation of the anterior cingulate cortex, an important pain center, in response to a painful rectal stimulus (Mertz et al., 2000).

Increasingly, evidence suggests a relationship between infectious enteritis and subsequent development of IBS. Inflammatory cytokines may play a role. In a survey of patients with a history of confirmed bacterial gastroenteritis (Neal et al., 1997), 25% reported persistent alteration of bowel habits. Persistence of symptoms may be due to psychologic stress at the time of acute infection (Gwee et al., 1999).

Recent data suggest that bacterial overgrowth in the small intestine may have a role in IBS symptoms. In one study (Pimentel et al., 2000), 157 (78%) of 202 IBS patients referred for hydrogen breath testing had test findings that were positive for bacterial overgrowth. Of the 47 subjects who had follow-up testing, 25 (53%) reported improvement in symptoms (ie, abdominal pain and diarrhea) with antibiotic treatment.

IBS may present with a range of symptoms. However, abdominal pain and altered bowel habits remain the primary features. Abdominal discomfort is often described as crampy in nature and located in the left lower quadrant, although the severity and location can differ greatly. Patients may report diarrhea, constipation, or alternating episodes of diarrhea and constipation. Diarrheal symptoms are typically described as small-volume, loose stools, and stool is sometimes accompanied by mucus discharge. Patients also may report bloating, fecal urgency, incomplete evacuation, and abdominal distention. Upper gastrointestinal symptoms, such as gastroesophageal reflux, dyspepsia, or nausea, may also be present (Lynn & Friedman, 1993).

Persistence of symptoms is not an indication for further testing; it is a characteristic of IBS and is itself an expected symptom of the syndrome. More extensive diagnostic evaluation is indicated in patients whose symptoms are worsening or changing. Indications for further testing also include presence of alarm symptoms, onset of symptoms after age 50, and a family history of colon cancer. Tests may include colonoscopy, computed tomography of the abdomen and pelvis, and barium studies of the small or large intestine.

Early Arthritis. The clinical presentation of different inflammatory arthropathies is similar early in the course of disease. As a result, it is often difficult to distinguish patients who are at risk of developing the severe and persistent synovitis that leads to erosive joint damage from those whose arthritis is more self-limited. Such distinction is critical in order to target therapy appropriately, treating aggressively those with erosive disease and avoiding unnecessary toxicity in patients with more self-limited disease. Current clinical criteria for diagnosing erosive arthropathies such as rheumatoid arthritis (RA) are less effective in early disease and traditional markers of disease activity such as joint counts and acute phase response do not adequately identify patients likely to have poor outcomes (Harrison & Symmons et al., 1998). Parameters reflective of the pathologic events occurring in the synovium are most likely to be of significant prognostic value.

Recent efforts to identify predictors of poor outcome in early inflammatory arthritis have identified the presence of RA specific autoantibodies, in particular antibodies towards citrullinated peptides, to be associated with erosive and persistent disease in early inflammatory arthritis cohorts. On the basis of this, a cyclical citrullinated peptide (CCP) has been developed to assist in the identification of anti-CCP antibodies in patient sera. Using this approach, the presence of anti-CCP antibodies has been shown to be specific and sensitive for RA, can distinguish RA from other arthropathies, and can potentially predict persistent, erosive synovitis before these outcomes become clinically manifest (Schellekens et al., 2000). Importantly, anti-CCP antibodies are often detectable in sera many years prior to clinical symptoms suggesting that they may be reflective of subclinical immune events ((Nielen et al., 2004; Rantapaa-Dahlqvist et al., 2003).

The clinical presentation of different inflammatory arthropathies is similar early in the course of disease. As a result, it is often difficult to distinguish patients who are at risk of developing the severe and persistent synovitis that leads to erosive joint damage from those whose arthritis is more self-limited. Such distinction is critical in order to target therapy appropriately, treating aggressively those with erosive disease and avoiding unnecessary toxicity in patients with more self-limited disease. Current clinical criteria for diagnosing erosive arthropathies such as rheumatoid arthritis (RA) are less effective in early disease and traditional markers of disease activity such as joint counts and acute phase response do not adequately identify patients likely to have poor outcomes (Harrison et al., 1998). Parameters reflective of the pathologic events occurring in the synovium are most likely to be of significant prognostic value.

Recent efforts to identify predictors of poor outcome in early inflammatory arthritis have identified the presence of RA specific autoantibodies, in particular antibodies towards citrallinated peptides, to be associated with erosive and persistent disease in early inflammatory arthritis cohorts. On the basis of this, a cyclical citrullinated peptide (CCP) has been developed to assist in the identification of anti-CCP antibodies in patient sera. Using this approach, the presence of anti-CCP antibodies has been shown to be specific and sensitive for RA, can distinguish RA from other arthropathies, and can potentially predict persistent, erosive synovitis before these outcomes become clinically manifest. Importantly, anti-CCP antibodies are often detectable in sera many years prior to clinical symptoms suggesting that they may be reflective of subclinical immune events (Nielen et al., 2004; Rantapaa-Dahlqvist et al., 2003).

Neuroinflammation. Neuroinflammation encapsulates the idea that microglial and astrocytic responses and actions in the central nervous system have a fundamentally inflammation-like character, and that these responses are central to the pathogenesis and progression of a wide variety of neurological disorders. This idea originated in the field of Alzheimer's disease (Griffin et al., 1989; Rogers et al., 1988), where it has revolutionized our understanding of this disease (Akiyama et al., 2000). These ideas have been extended to other neurodegenerative diseases (Eikelenboom et al., 2002; Orr et al., 2002; Ishizawa & Dickson, 2001), to ischemic/toxic diseases (Gehrmann et al., 1995; Touzani et al., 1999), to tumor biology (Graeber et al., 2002) and even to normal brain development.

Neuroinflammation incorporates a wide spectrum of complex cellular responses that include activation of microglia and astrocytes and induction of cytokines, chemokines, complement proteins, acute phase proteins, oxidative injury, and related molecular processes. These events may have detrimental effects on neuronal function, leading to neuronal injury, further glial activation, and ultimately neurodegeneration.

Neuroinflammation is a new and rapidly expanding field that has revolutionized our understanding of chronic neurological diseases. This field encompasses research ranging from population studies to signal transduction pathways, and investigators with backgrounds in fields as diverse as pathology, biochemistry, molecular biology, genetics, clinical medicine, and epidemiology. Important contributions to this field have come from work with populations, with patients, with postmortem tissues, with animal models, and with in vitro systems.

D. Pharmaceuticals

Pharmaceutically acceptable carriers that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrastenal, intrathecal, intrahepatic, intralesional and intracranial injection or infusion techniques. For localized disorders such as RA, the compositions will often be administered topically, e.g., in inflamed joints.

Sterile injectable forms of the compositions of this invention may be aqueous or an oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

The compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, the compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols. Such compositions are prepared according to techniques well-known in the art of pharmaceutical formulation.

The compositions of this invention may be administered topically, especially when the target of treatment includes areas or organs readily accessible by topical application, including diseases of the eye, the skin, the joints, or the lower intestinal tract. Suitable topical formulations are readily prepared for each of these areas or organs. Topical application for the lower intestinal tract can be effected in a rectal suppository formulation (see above) or in a suitable enema formulation. Topically-transdermal patches may also be used.

For topical applications, the compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the compositions may be formulated in an ointment such as petrolatum.

The compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

In one embodiment, the antibodies or therapeutic compounds of this invention may be incorporated into liposomes (“immunoliposomes” in the case of antibodies), alone or together with another substance for targeted delivery to a patient or an animal. Such other substances can include nucleic acids for the delivery of genes for gene therapy or for the delivery of antisense RNA, RNAi or siRNA for suppressing a gene in a T cell, or toxins or drugs for the activation of T cells through other means, or any other agent described herein that may be useful for activation of T cells.

In particular, applicants contemplate the use of lipid transport technologies described in U.S. Patent Publications 2001/0007666 and 2005/0136102, the contents of which are hereby incorporated by reference. Those documents disclose compositions and methods for transport or release of therapeutic and diagnostic agents or metabolites or other analytes from cells, compartments within cells, or through cell layers or barriers are described. The compositions include a membrane barrier transport enhancing agent and are usually administered in combination with an enhancer and/or exposure to stimuli to effect disruption or altered permeability, transport or release. In a particular embodiment, the compositions include compounds which disrupt endosomal membranes in response to the low pH in the endosomes but which are relatively inactive toward cell membranes, coupled directly or indirectly to a therapeutic or diagnostic agent. Other disruptive agents can also be used, responsive to stimuli and/or enhancers other than pH, such as light, electrical stimuli, electromagnetic stimuli, ultrasound, temperature, or combinations thereof. The compounds can be coupled by ionic, covalent or H bonds to an agent to be delivered or to a ligand which forms a complex with the agent to be delivered. Agents to be delivered can be therapeutic and/or diagnostic agents. Treatments which enhance delivery such as ultrasound, iontopheresis, and/or electrophereis can also be used with the disrupting agents.

In another embodiment, the antibodies or other compounds of the invention can be modified to improve its bioavailability, half-life in vivo, etc. For example, antibodies and other compounds can be pegylated, using any of the number of forms of polyethylene glycol and methods of attachment known in the art (see, e.g., Lee et al., 2003; Harris et al., 2003; Deckert et al., 2000).

For non-antibody compounds, the dose administered to a patient should be sufficient to effect a beneficial response in the subject over time. The dose will be determined by the efficacy of the particular modulators employed and the condition of the subject, as well as the body weight or surface area of the area to be treated. The size of the dose also will be determined by the existence, nature, and extent of any adverse side-effects that accompany the administration of a particular compound or vector in a particular subject. In determining the effective amount of the compound to be administered, a physician may evaluate circulating plasma levels of the compound, compound toxicities, and the production of anti-compound antibodies. In general, the dose equivalent of a compound is from about 1 ng/kg to 10 mg/g for a typical subject. Administration can be accomplished via single or divided doses.

According to another important embodiment of the present invention, the primary compounds may be administered in conjunction with one or more additional therapeutic agents, including agents normally utilized for the particular therapeutic purpose for which the antibody or compound is being administered, e.g. for treatment of autoimmune disease (anti-inflammatories, immunosuppressive agents) or cancer (chemotherapy, radiation, surgery, hormonal therapy). The other agents can either be administered together with the present antibody or compound, i.e., in the same pharmaceutical composition, or may be administered separately, including temporally. The additional therapeutic agent will generally be administered at a dose typically used for that agent in a monotherapy for the particular disease or condition being treated.

Further aspects and advantages of this invention are disclosed in the following experimental section, which should be regarded as illustrative and not limiting the scope of this application.

### X. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### NKG2D Expression by Large Proportions of Tumor Infiltrating and Circulating (Peripheral Blood) CD4 T⁺ Cells in Patients with MIC+ Tumors

Materials and Methods

Peripheral blood mononuclear cells and tumor infiltrating lymphocyte samples. Control peripheral blood was obtained from healthy volunteers who had given written informed consent in accord with protocols approved by the FHCRC review board. Peripheral blood mononuclear cells (PBMC) were isolated by density-gradient centrifugation (Ficoll/Hypaque, Pharmacia)

Previously isolated and liquN2 stored samples of tumor infiltrating lymphocytes (TIL) and matched PBMC from 26 tumor patients (6 breast, 8 lung, 4 ovarian, 4 colon cancers and 4 melanomas) typed for tumor-associated MIC expression and the presence of serum solMIC ( ) were included in this analysis.

Flowcytometry. PBMC and TIL were examined by three or four-color flow cytometry using various combinations of anti-CD3, -CD4, -CD8, -CD25, -CD27, -CD28, CD45RA, -CD45RO, -CD62, -CD103, -CD161, -CCR-7 (all BD Pharmingen), or anti-hGITR (R&D Systems) conjugated to phycoerythrin (PE), fluoresceinisothyocyanate (FITC) or PECy5 together with biotinylated anti-NKG2D (1D11) followed by allophycocyanine-conjugated streptavidin (Molecular Probes).

Results

It was previously shown that altered/diminished NKG2D expression on CD8⁺ T cells and NK cells in TIL and PBMCs (systemic) in solid tumor patients, which correlated with the presence of MIC expression by these tumors and resulted from ligand induced down-modulation of NKG2D.

More recent interest in the immunobiology of inducibly expressed NKG2D on CD4⁺ T cells (RA paper) prompted the inventors to revisit tumor setting and analyze NKG2D expression by tumor infiltrating (TIL) and matched peripheral blood CD4⁺ T cells in 26 (15 MIC+, 11 MIC−) of those previously characterized TL and PBMC sample pairs by monoclonal antibody (mAb) staining and flow cytometry. In contrast to control peripheral blood CD4⁺ T cells which are largely (˜98%) NKG2D negative, large proportions of CD4⁺ T cells both within tumors (range: 6.3-74.8, mean 20.6; n=15) and in the periphery (range: 6.6-71.8, mean 20.7; n=15) in patients with MIC+ tumors expressed NKG2D. The extent of NKG2D expression by CD4⁺ T cells did correlate with the extent of MIC expression by a given tumor (data not shown). In MIC− tumors, proportions of NKG2D⁺ CD4⁺ T cells were moderately increased (for TIL: range: 0.7-6.3, mean 3.4; n=11. For PBMC: range: 1.1-7.1, mean 3.4; n=11) compared to controls.

Previous results suggested that TCR-mediated T cell activation may lead to NKG2D expression on CD4⁺ T cells (Groh et al., 2003). T cell activation associated with any tumor immune responses thus may—at least in part—explain the increased frequencies of NKG2D⁺ CD4⁺ T cells in MIC− and MIC+ tumor patients. Consistent with this, a significant proportion of NKG2D CD4⁺ TIL and PBMC from both, MIC positive and negative tumor patients showed phenotypic signs of T cell activation and exhibited a memory phenotype as evidenced by multi-color flow cytometric analysis of CD25, CD27, CD28, CD45RA, CD45RO, CD62, and CCR-7 expression, respectively. Control NKG2D⁺ CD4⁺ T cells were negative for these markers. The more pronounced systemic increase in NKG2D-expressing CD4⁺ T cells in MIC+ tumor patients may result from MIC/NKG2D mediated proliferation and/or release of NKG2D-inducing cytokines (IL-15, TNFα, IL-10).

In MIC+ tumor patients, between 33-81% (mean: 54%, n=10) and 26-79% (mean: 50%) of NKG2D⁺ CD4⁺ TIL and PBMCs, respectively, were CD45RO⁺, between 46-79% (mean: 62%, n=10) and 27-85% (mean: 71%) of NKG2D⁺ CD4⁺ TIL and PBMCs, respectively, were HLA-DR⁺, and between 34-54% (mean: 44%, n=10) and 40-61% (mean: 51%) of NKG2D⁺ CD4⁺ TIL and PBMCs, respectively, were CD25⁺.

Significant increases (up to 70%) in NKG2D⁺ CD4⁺ TIL and PBMC in patients with MIC+ tumors. Moderate increases (mean ˜3.5%) in NKG2D⁺ CD4⁺ TIL and PBMC in patients with MIC− tumors. The true extent of 2D may even be higher, there may be a ying and yang between inducing/proliferating and down modulating signals, and so ex vivo results may reflect balance between induction/ligand-induced down-modulation. Large proportions of 2D⁺ CD4⁺ TILs and PBMCs, regardless of their origin, have activated (CD25, HLA-DR) and memory (CD45RO) phenotype.

### Example 2

### Activated CD4 T Cell Subsets Express NKG2D and Proliferate in Response to MIC Engagement While Bystander NKG2D⁻ CD4⁺ T Cells are Growth Inhibited

Materials and Methods

CD4 T cell stimulation. CD4 T cells were purified (>99% purity) from PBMC using CD4 MicroBeads (Miltenyi Biotec GmbH) according to the manufacturer's instructions and tested for purity by flow cytometry.

For the analysis of NKG2D expression in activated CD4 T cells, freshly isolated pure CD4⁺ T cells were plated at 0.3×10⁶/2 ml AIM-V medium (Gibco)/well in 24 well plates for various periods of time with or without the presence of mitogenic stimuli and/or the presence of NKG2D ligands. Mitogenic stimuli included either cross-linked anti-CD3 (plates were first coated with AffiniPure F(ab′)₂ fragment goat anti-mouse IgG1, Fcγ fragment specific (Jackson ImmunoResearch Laboratories, Inc.) at 10 μg/ml in PBS over night at 4° C. followed by a 4 hr room temperature incubation with anti-CD3 (OKT3, Orthoclone, Ortho Biotech Products, L.P.) at 50 ng/ml (if not indicated otherwise), PHA (5 μg/ml), or PMA/Ionomycin. Ligand additions consisted of either irradiated CLR-MICA and control transfectants at a T cell:transfectant ratio of 5:1, or recombinant soluble MICA at 25 ng/ml if not indicated otherwise. Cells were harvested at various time points after the initiation of culture and tested for NKG2D, CD4 and CD3 coexpression using directly conjugated mAbs for standard three color flow cytometry.

For analysis of CD4⁺ T cell proliferation in conjunction with NKG2D expression, 1×10⁷ freshly isolated pure CD4⁺ T cells/ml PBS were labeled with an equal volume of 2 μM CFSE (Molecular Probes) for 8 min at room temperature. CFSE was quenched with an equal volume of fetal calf serum for one min followed by three washes with AIM-V medium. CFSE labeled cells were then plated, stimulated and phenotypically analysed as described above. Additional ligands tested in this experimental setting included any of either C1R-MICB, ULBP-1, -2, -3, 4, or -5 transfectants, recombinant soluble MICB, or 5% soluble MIC containing serum from a MIC+ tumor patient and MIC− control serum. To test ligand specificity, ligand containing cultures were treated with either 10 μg/ml 6D4 (anti-MICA/B), 10 μg/ml 3F1 (anti-ULBP-1), or appropriately diluted isotype control immunoglobulins. In some experiments, ligand was replaced by solid-phase anti-NKG2D (1D11) at the concentrations ranging from 5-50 ng/ml. In some experiments, ligand/solid-phase mAb were added on day 1, 2 or 3 after the initiation of culture.

To assess inhibition of CD4⁺ T cell proliferation by standard [³H]thymidine incorporation experiments, freshly isolated pure CD4⁺ T cells were plated at 1×10⁵ cells/well in a 96 well flat bottom plate containing titrated cross-linked anti-CD3 (see above) or isotype control Ig with or without titrated recombinant soluble MIC. After 72 hrs at 37° C., plates were pulsed with 3 μCi of [³H]thymidine (Perkin Elmer) for 12 hrs. Plates were harvested and samples analysed by liquid scintillation using Uni-Filter GF/C plates and a TopCount Counter (both from Packard). Counts from triplicates were averaged.

Results

To identify factors responsible for the phenotypic conversion of large numbers of CD4⁺ T cells in MIC+ tumor patients, purified CD4⁺ peripheral blood T cells from healthy donors were cultured in the presence or absence of activation signals such as crosslinked anti-CD3, PHA, or PMA/Ionomycin with or without either cell associated or soluble MIC ligands for several days. NKG2D expression by CD4⁺ T cells was followed by flowcytometry using biotin conjugated 1D11 and PE-streptavidin. After 24 hrs, the proportion of NKG2D⁺ CD4⁺ T cells more than doubled in activated but not in control cultures and continued to increase for up to a week. All activation stimuli tested had a similar effect.

For the first three days of culture, the presence of MIC had no apparent effect on the emergence of NKG2D-expressing CD4⁺ T cells. However, beginning with day four or five, both membrane-bound and soluble MIC-containing cultures contained substantially larger percentages of NKG2D⁺ CD4⁺ T cells than those without ligand. This increase in the proportion of NKG2D⁺ CD4⁺ T cells in MIC containing cultures could result from either MIC/NKG2D costimulated proliferation or cytokine release followed by NKG2D induction. To differentiate between these two possibilities, ex vivo purified CD4⁺ T cells were labeled with 5-(and-6)-carboxy fluorescein diacetate succinimidyl ester (CFSE) prior to activation and, after staining for NKG2D, tested for both proliferation and NKG2D expression on days 4 or 5 of culture. As for the previous experiments, MIC was added in either cell surface expressed or soluble form, which had indistinguishable effects. Although, by flow cytometric analysis, unlabeled MIC-transfectants were clearly separable from CFSE labeled CD4⁺ T cells, more easily interpretable results were obtained in the presence of soluble MIC and chosen for documentation. In the absence of activation stimuli, CD4⁺ T cells did not proliferate and proportions of NKG2D⁺ CD4⁺ T cells remained unchanged over time in culture. As expected, within four to five days of T cell activation, proportions of NKG2D-expressing T cells were increased and a fraction of CD4⁺ T cells including NKG2D⁺ expressers showed signs of proliferation by undergoing several cell divisions. A dramatically different and partially unexpected CFSE-NKG2D double-labeling profile emerged from dot blots obtained with CD4⁺ T cell cultures exposed to anti-CD3 and MIC with NKG2D⁺ and NKG2D⁻ populations displaying containing proliferation behaviors, right dot blot and histogram). Proliferation of NKG2D⁺ cells was enhanced compared to ligand free controls. Thus, as with CD8⁺ T cells, ligand engagement of NKG2D on CD4⁺ T cells co-stimulated proliferation of these cells and thus may explain the marked increase of NKG2D⁺ CD4⁺ T cells in MIC+ tumor patients (chronic stimulation can lead to large proportions). In contrast, proliferation of NKG2D⁻ T cells was dramatically inhibited compared to ligand free cultures, with only a small subpopulation of cells undergoing cell divisions. The extent of this inhibitory effect was dose dependent as revealed by ³H thymidine incorporation experiments of purified CD4⁺ T cells exposed to titrated concentrations of the anti-CD3 mAb and soluble MIC.

To assess whether NKG2D⁻ CD4⁺ T cell growth inhibition as evidenced by CFSE profiles could result from either cell death or cell cycle arrest. Annexin-V staining of anti-CD3 activated control and ligand/solid-phase anti-NKG2D mAb containing CD4⁺ T cell bulk cultures gave identical results suggesting that apoptosis did not account for the observed inhibition of proliferation of NKG2D⁻ CD4 T cells. However, cell cycle analysis using propidium iodide (P) staining revealed an accumulation of the majority of cell in G₀/G₁ in ligand/solid-phase anti-NKG2D mAb exposed cultures whereas significant proportions of control anti-CD3 stimulated CD4 T cells entered the S and G₂/M phase within five days of culture.

Growth inhibition of the NGK2D⁻ CD4⁺ T cells subset in activated partially NKG2D⁺ CD4⁺ T cell bulk cultures also occurred in the presence of cell surface expressed and soluble MICB, patient serum containing soluble MIC as well as C1R transfectants expressing either one of the ULBP-1 through-5 NKG2D ligands and was neutralized in the presence of mAbs specific for MICA/B or ULBP-1 through 5, respectively. Addition of solid-phase anti-NKG2D could substitute for the presence of NKG2D ligands which indirectly suggested that all ligand mediated effects resulted from ligand mediated NKG2D triggering.

In summary, these experiments showed that mitogenic stimuli induce NKG2D expression in a subset of CD4⁺ T cells, which will proliferate upon ligand engagement. Phenotypic conversion of CD4⁺ T cells observed in MIC+ tumor patients may represent in vivo equivalent of this in vitro observation. In CD4⁺ bulk cultures, MIC/NKG2D costimulated expansion of NKG2D⁺ CD4⁺ T cells was paralleled by an inhibition of proliferation of NKG2D⁻ CD4⁺ T cells.

Inhibitory effect seen with all NKG2D ligands, neutralized by respective mAbs, ligands can be substituted by solid-phase anti-NKG2D.

### Example 3

### Growth Inhibition of NKG2D⁻ T Cells in Mixed NKG2D⁺ and NKG2D⁻ CD4⁺ T Cell Bulk Cultures is Mediated by Soluble Factors Released by NKG2D⁺ CD4 T Cells Upon NKG2D-Ligand Engagement

Materials and Methods

To simplify the description of the experimental set up chosen to define the nature of cells mediating growth inhibition of NKG2D⁻ CD4⁺ T cells, these cell populations will from now on be referred to as stimulator and responder cells, respectively.

Responder cells were freshly isolated CD4⁺ T cells depleted of their NKG2D⁺ subset using PE-anti-NKG2D (1D11) and Anti-PE MicroBeads (Miltenyi Biotech) according to the manufacturer's instructions prior to CFSE labeling plated on solid-phase OKT3 as described above. Stimulator cells included either one of the following cell populations: 1) autologous purified NKG2D depleted CD4⁺ T cells cultured for three days in AIM-V which did not result in NKG2D induction. These cells are thus referred to as uninduced stimulators. 2) autologous purified NKG2D-depleted CD4⁺ T cells cultured for three days in AIM-V and solid-phase OKT3 which did result in NKG2D induction. These cells are thus referred to as induced stimulators. 3) induced stimulators depleted of the NKG2D⁺ subset as described above. 4) NKG2D⁺ CD4⁺ T cells purified from induced stimulators using PE-1D11 and cell sorting on a BD FACSAria. This purification approach was chosen to avoid NKG2D crosslinking prior to the addition of ligand. 5) NKG2D⁺ and NKG2D CD4⁺ T cell lines which were established from sorted populations. Stimulator cells were added to responders at a previously optimized cell:cell ratio of 1:5 either directly or to the upper chamber of transwells with 0.4 μm pore size membranes in the presence or absence of MIC beads. MIC coated beads was used as ligand source since it guaranteed confinement of ligand to the upper chamber, whereas soluble ligand or ligand shed from transfectants would have entered the bottom chamber.

Experiments with solid-phase anti-NKG2D in place of ligand additions confirmed NKG2D triggering as the critical factor in mediating growth inhibition of NKG2D⁻ T cells, (participation of ligand binding to a putative non-NKG2D receptor could not be excluded). Purities of freshly isolated and activated CD4 bulk cultures consistently exceeded 99% of total cells with the remaining cells representing NKG2D⁻ CD4^{low} CD3⁻ presumably monocytes. Thus, CD4⁺ NKG2D⁺ T cells did represent the most likely candidates for mediating the CD4⁺ NKG2D⁻ T cell growth inhibition. This was supported by experiments where ligand or solid-phase anti-NKG2D were added to activated CD4⁺ T cells over time so that the extent of inhibitory effects on NKG2D CD4⁺ T cells could be measured in the context of the increasing extent of NKG2D induction on CD4⁺ T cells in these cultures. Ligand/anti-NKG2D added on day 1 or 2 of culture, with NKG2D expression increased compared to uncultured day 0 conditions, was significantly more effective in inhibiting NKG2D T cell proliferation than ligand/mAb present since the initiation of culture (data not shown, could be shown). Direct evidence as to the identity of cells responsible for inhibiting NKG2D⁻ CD4⁺ T cell proliferation, however, should come from testing phenotypically homogeneous, i.e., pure NKG2D⁺ CD4⁺ or NKG2D⁻ CD4⁺, respectively, cell populations for their inhibitory potential. Optimally designing such experiments was dependent on whether growth arrest of NKG2D⁻ CD4⁺ T cells required cell-cell contact or was dependent on soluble factors.

Results

To test this, 48 or 72 hr anti-CD3-stimulated, and thus NKG2D-induced, or unstimulated NKG2D⁻ CD4⁺ T cell bulk cultures, were mixed with MICA coated sepharose beads as a source of stably bound ligand. The cell/bead mix was then added either directly to autologous CFSE-labeled freshly purified NKG2D⁻ CD4⁺ T cells plated on solid phase anti-CD3 or to upper chambers of transwell plates separated from CFSE-labeled freshly purified solid-phase stimulated NKG2D⁻ CD4 T⁺ cells by 0.4 μm pore size membranes. Strong growth inhibition of CFSE labeled cells occurred in cultures treated with anti-CD3 stimulated NKG2D induced cells in the presence of MICA beads independent of whether cell-cell contact was permitted or not suggesting that soluble factors rather than cell-cell contact were mediating inhibition. Cultures treated with uninduced CD4⁺ NKG2D⁻ cells and MICA beads also revealed growth inhibition, most likely resulting from MIC ligation of NKG2D induced in both CFSE labeled and added CD4⁺ T cells as a result of T cell activation. Consistent with this, physical separation of stimulator cells and MIC beads from responder cells, had no inhibitory effect. Thus, the transwell setting was suitable (and ideal since reinduction of depleted cells could be avoided) to extend these experiments to using phenotypically pure stimulator cell populations such as anti-CD3 activated CD4⁺ bulk populations depleted of the NKG2D-induced subset, NKG2D-induced CD4⁺ T cells purified from such cultures as well as NKG2D⁺ or NKG2D⁻ CD4⁺ T cell clones. Confirming all earlier indirect results, NKG2D⁺ but not NKG2D⁻ CD4⁺ T cells were able to mediate responder cell growth inhibition upon ligand engagement.

### Example 4

### CD4⁺ Suppression of Immune Response is Mediated by FasL

Materials and Methods

Amounts of sFas ligand in 24 hrs supernatants CD4 T cells stimulated with anti-CD3 together with or without ligand/solid-phase anti-NKG2D were tested by commercial ELISA with matched antibody pairs in relation to standard pairs (R&D Systems). The same supernatants were tested for their ability to induce cell death in Jurkat cells in the presence or absence of the non-cytotoxic blocking anti-Fas mAb SM1/23.

Results

No evidence for increased Foxp3 in the presence of ligand was found. Also, cell-cell contact was not required, so if inhibition is mediated by regulatory T cells, they would rather fall into the category if Tr1 cells. Indeed, ligand engagement of NKG2D⁺ CD4⁺ T cells did result in the secretion of Tr1 cytokines such as IL-10 and TGF. Thus, NKG2D⁺ CD4⁺ T cells could either function as Tr1 cell themselves or through cytokine release indirectly support expansion of Tr1 cells. Efforts to neutralize using anti-IL-10, anti-ILR mAb as well as anti-TGFβ, alone or in combination had no effect. Another candidate mediator of growth arrest was FasL. Indeed, FasL can be measured in CD4⁺ cultures (anti-CD3+/− ligand-solidphase anti-NKG2D) by ELISA and SN of these cultures have Jurkat cytotoxic effect. Most importantly, the presence of non-stimulatory anti-Fas mAb resulted in neutralization of the inhibitory effect. Neutralization was rather complete, so although contribution of other factors (Tr1, Apo2L) can not be excluded, the main mediator appears to be FasL/Fas growth arrest.

### Example 5

### NKG2D-Dependent Immunosuppression and Mechanism of MICA Shedding

Epithelial tumor shedding of soluble MIC ligands causes NKG2D receptor endocytosis and degradation and thus systemic downmodulation on NK cells and CD8 T cells. As a consequence, NK and T cell responsiveness is impaired. Most CD4⁺ T cells are normally negative for NKG2D. In cancer patients with tumor-associated MIC, however, variable proportions of CD4⁺ T cells, ranging from about 8 up to 77% (mean 25% among 28 individuals) express low to very high levels of NKG2D. In vitro activation results in a rapid induction of NKG2D on about 8 to 12% of normal CD4 T cells, which proliferate in the additional presence of solid-phase anti-NKG2D mAb or soluble MICA providing costimulation. Over an extended culture period, these T cells expand substantially, and cause growth arrest of NKG2D⁻ CD4⁺ T cells by secretion of soluble mediators. These preliminary results reveal an additional immunosuppressive effect of MIC-NKG2D in tumor settings.

The shedding of soluble MIC is thought to be mediated by metalloproteinases. However, recent evidence has unraveled more complex molecular events. On the surface of tumor cell lines and freshly isolated tumor cells, MICA specifically associates with ERP5, an endoplasmic reticulum protein of the protein disulphide isomerase (PDI) family. Pharmacological inhibition of thioreductasse catalytic activities and si-RNA-mediated silencing of ERP5 expression profoundly reduce shedding of soluble MICA and interfere with its physical interaction with ERP5. Reduction of an intradomain disulphide bond of MICA causes a conformational destabilization that is a necessary prerequisite for proteolytic cleavage within the peptide sequence that connects the proximal α3 and transmembrane domains.

### Example 6

### Disulfide Isomerase-Enabled Shedding of Tumor-Associated NKG2D Ligands

Materials and Methods

Tumor samples and cell lines, antibodies, tetramers, pharmacological inhibitors, and ELISA for sMICA. Source of tumor cell suspensions has been reported. Cell lines were from the American Type Culture Collection. Anti-NKG2D (mAb 1D11), anti-MICA (mAb 2C10), anti-MICA and anti-MICB (mAb 6D4) have been described previously. Rabbit polyclonal anti-ERP5 and anti-GRP78 were from Abcam and Stressgen, respectively. Recombinant MICA*001 (residues 1-276) and ULBP2 (residues 1-202) were produced in transfected 293T cells and purified from culture supernatant using Invitrogen methodology. Tetramers were prepared by conjugation with phycoerythrin (PE)-streptavidin. Cells were stained with saturating tetramer concentrations for 1 h at 4° C. and examined by flow cytometry. Non-glycosylated MICA was expressed in bacteria and purified as described previously. Bacitracin, DTNB, and PAO were from Sigma. Metalloproteinase inhibitors GM 6001 and MMP Inhibitor III were from Calbiochem. The ELISA for sMICA has been described elsewhere.

Identification of MICA-binding surface proteins. U266 and U937 cells (each 5×10⁹) were dounce-homogenized in 10 mM Tris-HCl (pH7.6), 0.5 mM MgCl₂, 1 mM PMSF, 1 μg/ml leupeptin, and 1 μg/ml pepstatin. Membrane fractions were isolated from cleared supernatants by dextran-PEG partitioning, washed [8% sucrose, 5 mM Tris-HCl (pH 7.4)], and dissociated in lysis buffer [50 mM Tris-Cl (pH 7.4), 1% Triton X-114, 150 mM NaCl, 5 mM EDTA, 5 mM iodoacetamide, protease inhibitors]. Cleared supernatants were warmed to 37° C. and proteins partitioned during Triton X-114 phase separation. Proteins in aqueous fractions were affinity-purified using MICA-conjugated sepharose beads, visualized by SDS-PAGE and silver staining, and analysed by MALDI-TOF at the FHCRC Mass Spectrometry Facility. For immunoblotting, MICA-binding proteins were prepared after cell surface biotinylation with EZ-Link Sulfo-NHS-LC-Biotin (Pierce).

siRNA expression and real-time PCR. Oligonucleotide pairs for siRNA-17 and siRNA-19 targeting ERP5 (disulfide isomerase-related protein P5; GenBank accession number D49489) mRNA at positions 316-338 and 556-567 were GATCTTGTTGTCAAAGTTGGTGCAGTTGTCTTCTTCTCAACTGCACCAACTTTGACAA CATTTTTG (SEQ ID NO:5) and AATTCAAAAATGTTGTCAAAGTTGGTGCAGTTG AGAAGAAGACAACTGCACCAACTTTGACAACAA (SEQ ID NO:6), and GATCTTGATAGTTCAAGTAAGAAGGATGTCTTCTTCTCATCCTTCTTACTTGAACTAT CATTTTTG (SEQ ID NO:7) and AATTCAAAAATGATAGTTCAAGTA AGAAGGATGAGAAGAAGACATCCTTCTTACTTGAACTATCAA, (SEQ ID NO:8) respectively (all 5′-3′; internal hairpin sequence, 3′-end termination signal, and Bgl II and Eco RI overhangs are underlined). Annealed primers were ligated into retroviral vector pBABE-GFP and constructs sequenced. Virus was produced in Phoenix amphotropic packaging cells and culture supernatant used for infection of A375 cells, which were sorted for GFP expression. Real-time RT-PCR was performed as described previously, using primer sets TGCGGCACGCTGCAGGGCT (SEQ ID NO:9) and TTGACAGTGACCACACCATGGAGCATA (SEQ ID NO:10) for ERP5, and GGAACGGAAAGGACCTCAGGATG (SEQ ID NO:11) and CTGGGAGCTCCTGGTGCTGTTG (SEQ ID NO:12) for MICA cDNA, and SYBR Green reagents (Molecular Probes).

Preparation of sRNase and capturing of ERP5-MICA complexes. RNase A was denatured and reduced in 0.1M Tris-OH (pH 8.6), 6 M guanidine hydrochloride, and 0.15M dithiothreitol for 24 h at room temperature (RT), and desalted using D-Salt Dextran columns (Pierce) equilibrated with phosphate-buffered saline (PBS). 2×10⁶ semi-confluent Hela cells were exposed to various concentrations of sRNase for 16 h, washed, and surface biotinylated with EZ-Link Sulfo-NHS-LC Biotin. Labeled cells were incubated in 0.5 ml 10% (w/v) TCA in PBS for 30 min on ice, washed sequentially in 10% and 5% TCA in PBS, and lysed in 50 mM Tris (pH 7.4), 1% Surfact-Amps NP-40 (Pierce), 150 mM NaCl, 5 mM EDTA, 40 mM N-ethylmaleimide (Sigma), 1 mM PMSF, leupeptin (1 μg/ml), and pepstatin (1 μg/ml). Lysate pH was adjusted to 7.0 with 1M Tris-OH (pH 9.5). Protein complexes were precipitated with mAb 2C10 (anti-MICA) or ERP5 polyclonal antibody, treated with N-glycanase, and processed for SDS-PAGE. For sequential precipitation, mAb 2C10 immunocomplexes were dissociated with 150 mM Tris (pH 7.4), 0.5% SDS, and 10 mM DTT, diluted 10-fold with lysis buffer containing 25 mM iodoacetamide, incubated for 1 h at RT for DTT neutralization and sulfhydryl alkylation, and reprecipitated with anti-MICA mAb BAMO-1 (Axxora) or anti-ERP5. For determination of sMICA C-terminal cleavage, supernatant from C1R-MICA transfectants grown in Opti-MEM (Gibco) was concentrated using Amicon Ultra-15 centrifugal filters (Mllipore). sMICA was immunoprecipitated, treated with N-glycanase, isolated by SDS-PAGE, and subjected to peptide fragmentation analysis by MALDI mass spectrometry at the Harvard University Microchemistry Facility.

ERP5 activity assays. Ectodomain-only MICA, Siderocalin, and CD94-NKG2A were expressed in bacteria and purified as described previously. The inventors similarly produced ERP5 (residues 1-421 of the mature protein), the ERP5 fragments 1-118 and 135-421, the C36S and C39S mutants (made by Stratagene Quick Change methodology) of ERP5 1-118, and the isolated MICA α1α2 platform (residues 1-180) and α3 domains (residues 187-274). All ERP5 sequences were fused to N-terminal hexahistidine tracts and included a C-terminal stop codon to prevent expression of the adjacent hexahistidine in pET22(b). Recombinant proteins were purified by metal affinity (BD Talon, Clontech) and size exclusion (Superdex 200, Pharmacia) chromatography. For testing of functional activity, ERP5 or derivative proteins (2 μg) were incubated at RT with MICA substrates or control proteins (1.5 μg) in PNEA [25 mM PIPES (pH 7), 150 mM NaCl, 1 mM EDTA, and 0.02% sodium azide] in a total volume of 5 μl per time point sample, mixed with 2×SDS-PAGE sample buffer (5 μl) with or without β-ME and resolved in 15% Tris-glycine or 12% Bis-Tris NuPAGE (Invitrogen) gels.

Results

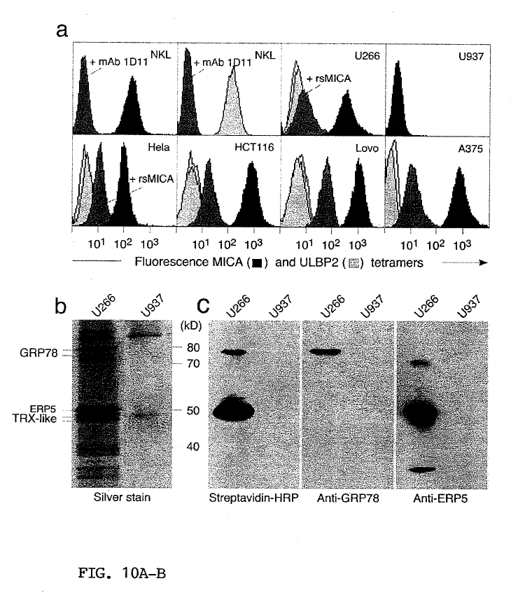

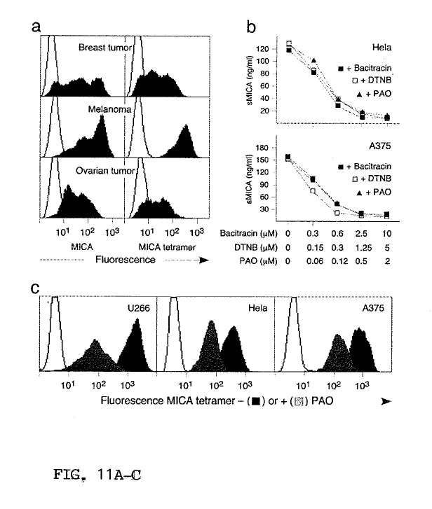

Randomly oligomerized recombinant sMICA (rsMICA) or other NKG2D ligands produced as immunoglobulin fusion proteins exclusively bind to NKG2D expressing lymphocytes. Testing MICA and ULBP2 ligand tetramers, the inventors confirmed that these high avidity reagents stained the NKL NK cell line and that binding was exclusively accounted for by NKG2D as indicated by the complete inhibitory effect of antibody masking (FIG. 10A). However, by flow cytometry screening of ˜40 cell lines, the inventors observed that MICA, but not ULBP2, tetramers stained cell types lacking NKG2D. The highest fluorescence intensities were recorded with U266 myeloma cells and all of 10 epithelial tumor lines tested and correlated with relatively large amounts of cell surface MICA. With fibroblast and lymphocyte cell lines that included other myeloid cells, B cells, and NKG2D⁻ T cells, the staining intensities were typically more modest and heterogeneous. Only monocytic U937 cells were identified as negative for tetramer binding (FIG. 10A). The MICA tetramers were prepared using highly glycosylated protein secreted by transfected human 293T cells. However, tetramer binding was not reduced after N-glycanase-mediated cleavage of polypeptide-linked carbohydrates on U266 and epithelial tumor cells but was inhibited in the presence of unglycosylated rsMICA produced in bacteria (FIG. 10A; data not shown). Thus, these results revealed an unrecognized interaction involving MICA, but not NKG2D ligands in general, and a presumably proteinaceous cell surface factor.

Candidate tetramer-binding proteins were purified from U266 and negative control U937 cell outer membrane fractions by affinity chromatography using MICA-coupled sepharose beads. SDS-polyacrylamide gel electrophoresis (SDS-PAGE) and silver staining revealed two sets of protein bands that were detected with U266 but not U937 cells (FIG. 10B). By mass spectrometry, two protein bands in the 76-78 kilodalton (kD) molecular mass range unambiguously corresponded to glucose-regulated protein 78 (GRP78, also known as BiP). A major protein band of 50 kD was identified as ERP5 (also known as P5), and two additional proteins of about 47 and 48 kD shared similarities with thioredoxin family members. Because all of these proteins are typically intracellular, the inventors scrutinized their outer cell membrane localization. However, employing the same purification protocol and surface-biotinylated cells, immunoblots probed with streptavidin-horse radish peroxidase (HRP) or polyclonal antisera confirmed the presence of GRP78 and more prominently of ERP5 on the surface of U266 but not U937 cells (FIG. 10C). ERP5 is related to protein disulfide isomerase (PDI). Both proteins contain two thioredoxin-like domains, each with a pair of active site cysteines in CXXC motifs, and mediate the intracellular formation of nascent polypeptide disulfide bonds; however, they have also been implicated in extracellular disulfide exchange.

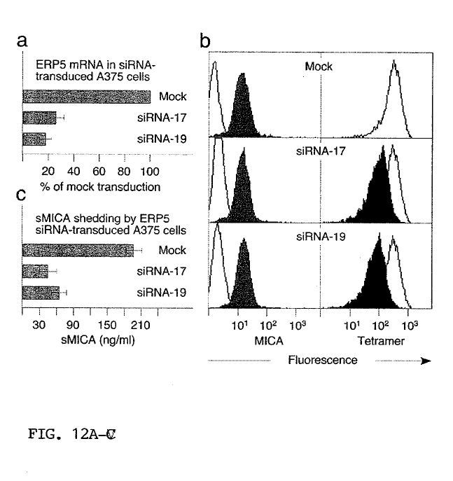

In exploring a functional relationship between MICA and ERP5, the inventors were guided by the epithelial tumor-associated expression that is characteristic of MICA but not of the ULBP family of NKG2D ligands. This idea was encouraged when freshly isolated epithelial tumor cells displayed similar patterns of tetramer and anti-MICA antibody binding (FIG. 11A). The inventors therefore tested a potential role of ERP5 in MICA shedding by exposing U266 cells and epithelial tumor cell lines (Hela, A375 melanoma, and HCT116 and Lovo colon carcinoma) to bacitracin, an antibiotic that inhibits PDI oxidoreductase activities, or to DTNB [5,5-dithiobis-(2-nitrobenzoic acid)] or PAO (phenylarsine oxide), which impair PDI function by forming disulfide and coordination bonds, respectively, with thiol groups in its catalytic sites. All inhibitors markedly reduced the production of sMICA in a concentration-dependent manner (FIG. 11B; data not shown). Moreover, treatment with PAO also diminished tetramer binding, suggesting that MICA interacts directly with an ERP5 catalytic site (FIG. 11C). To obtain complementary evidence and preclude an involvement of thiol isomerases other than ERP5, we expressed siRNA constructs targeting two regions of ERP5 mRNA in retrovirally-transduced A375 cells. As measured by real-time reverse transcription PCR (RT-PCR), ERP5 mRNA was reduced by about 70-80% (FIG. 12A). As a consequence, MICA tetramer binding and sMICA shedding decreased substantially, although the amounts of MICA mRNA and surface protein were not noticeably changed (FIGS. 12B-C; data not shown).

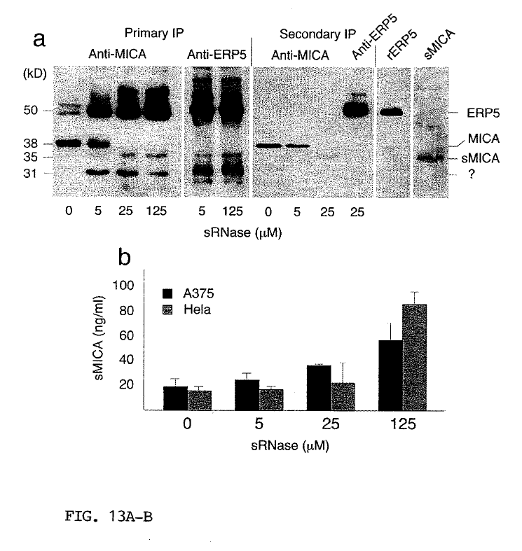

The apparent functional association between ERP5 and MICA was biochemically analysed. Initial failure to co-immunoprecipitate interacting proteins from Hela cell lysates implied that ERP5 and MICA form no stable complexes, possibly due to transitory disulfide exchange. However, ERP5 specifically co-immunoprecipitated with MICA when Hela cells were treated with trichloroacetic acid (TCA) to trap mixed disulfide polypeptides and quench thiol interchange (FIG. 13A, lane 1). Sulfhydryl groups in cell lysates were alkylated to prevent artifactual formation of disulfide bonds and immunocomplexes deglycosylated with N-glycanase. This procedure was modified by using Hela cells grown in the presence of ‘scrambled’ (denatured and reduced) RNase (sRNase), which served as excess substrate shifting ERP5 equilibrium towards the reduced state, thereby favoring disulfide exchange with MICA. Analysis by reducing SDS-PAGE showed that increasing concentrations of sRNase resulted in larger amounts of ERP5 co-immunoprecipitating with MICA (FIG. 13A, lanes 14). Concomitantly, the MICA polypeptide of 38 kD (shortened in Hela cells due to homozygous cytoplasmic tail deletion) disappeared, and proteins with molecular masses of 31 and 34 kD emerged that were not detected in the absence of sRNase. Of these two proteins, the 34 kD species corresponded to truncated sMICA as determined by secondary precipitation from dissociated immunocomplexes and comparison to sMICA isolated from Hela cell culture media (FIG. 13A, lanes 3, 4, 9 and 12). Although the identify of the 31 kD protein could not be determined, it may represent another substrate that was recruited into ERP5-MICA complexes. Similar data were obtained using anti-ERP5 for immunoprecipitations (FIG. 13A, lanes 5 and 6). Thus, these results demonstrated dynamic interactions between ERP5 and MICA that were closely tied to the production of sMICA, which was corroborated by large increases of sMICA in sRNase-treated Hela and A375 cell cultures (FIG. 13B).

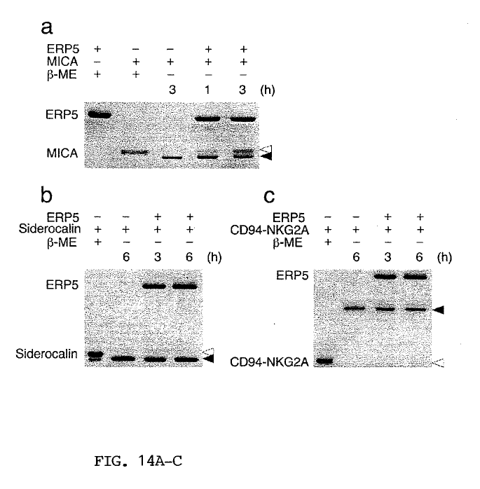

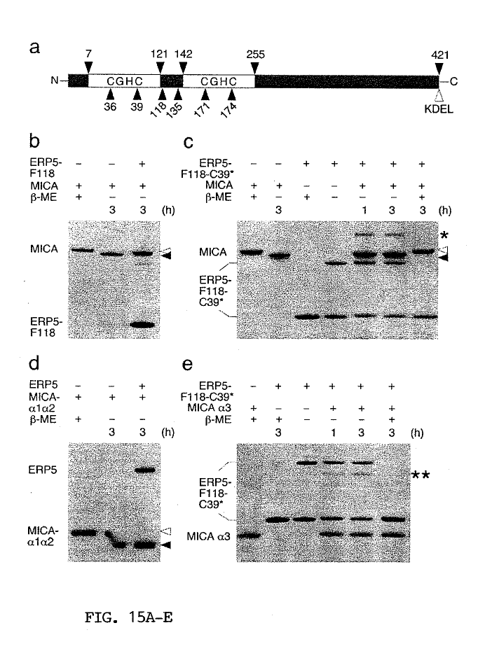

To demonstrate ERP5-mediated MICA disulfide bond reduction and explore substrate and domain specificities, bacterially produced and purified recombinant proteins were mixed and incubated in the absence of reducing agents and thus under oxidizing conditions. Non-reducing gel electrophoresis and comparison to control β-mercaptoethanol (β-ME)-treated and untreated samples showed gradual reduction of MICA (FIG. 14A). This was remarkable as ERP5 affected an intact, properly folded, substrate protein isoenergetically and in the absence of any other factor in solution. A similar result was obtained with the closely related MICB (data not shown). In contrast, ERP5 did not affect unrelated proteins with relatively accessible intrachain (Siderocalin) or intrachain and interchain (CD94-NKG2A) disulfide bonds (FIGS. 14B-C). No synergistic effect was observed when MICA was exposed to ERP5 together with GRP78. Of the two ERP5 thioredoxin-like domains, which were expressed as two separate polypeptides (amino acid residues 1-118 and 135-421; FIG. 15A), only the N-terminal domain displayed functional activity (FIG. 15B; data not shown). As with PDI, ERP5 employs a catalytic mechanism whereby one active site cysteine invades the target disulfide, transiently forming a disulfide-linked heterodimer which is resolved by disulfide exchange with the second active site cysteine. Of two ERP5 1-118 mutant figments, C36S showed no activity on MICA substrate whereas C39S formed a trapped disulfide-linked intermediate, thus confirming the role of C36 as the invading and C39 as the resolving cysteine in this reaction (FIG. 15C; data not shown). By size exclusion chromatography, intact ERP5 was a trimer in solution whereas the two individual domains behaved as monomers (data not shown). Thus, ERP5 multimerization by interdomain interactions was not required for MICA substrate reduction.

Similar to conventional MHC class I molecules, MICA contains three intrachain disulfide bonds located between amino acid residues 36 and 41, 96 and 164, and 202 and 259 in the α1, α2, and the C-type immunoglobulin-like α3 domain, respectively. To identify the target disulfide, the α1α2 platform and α3 membrane-proximal domains were expressed and tested separately. ERP5 displayed no catalytic activity with the α1α2 domain (FIG. 15D). Because we were unable to electrophoretically resolve reduced and non-reduced forms of the relatively small α3 domain, we used the ERP5 1-118 polypeptide fragment with the C39S mutation for analysis. Gel electrophoresis revealed a large protein band shift corresponding to an unresolved mixed disulfide heterodimer (FIG. 15E). Thus, the disulfide bond targeted by ERP5 was in the MICA α3 domain.

Proteolytic cleavage of MICA is thought to be mediated by metalloproteinases. However, the inventors observed no reduction in sMICA shedding by metalloproteinase inhibitors, suggesting that diverse proteases may have the ability to cleave MICA. To determine the MICA cleavage site, they purified sMICA from transfectant C1R-MICA cell cultures, which express modest amounts of ERP5 but proliferated vigorously in serum-free media in which U266 and epithelial tumor cells grew poorly. C-terminal sequencing by mass spectroscopic analysis of tryptic peptide fragments revealed ragged MICA C-termini composed of several neighboring amino acid residues at or near the transmembrane boundary.

In conclusion, these results reveal a dynamic interaction between surface ERP5 and the transmembrane-anchored NKG2D ligand MICA, whereby transitory disulfide exchange concurrent with remodeling of the MICA α3 domain enable sMICA shedding. The scale of this conformational change, affecting a very stable protein fold in an isoenergetic process, is likely large enough to completely change the accessability of the MICA membrane-proximal region to proteolytic activity after mixed disulfide resolution. A potential role of MICA-associated surface GRP78 in assisting this reaction remains uncertain. Escape from intracellular retention of proteins such as ERP5 and PDI and their surface attachment are not well understood but may simply involve leakage after increased expression associated with proliferation and cellular stress—conditions that also result in induced expression of MICA in permissive types of cells. Precedent for biological functions of surface thiol isomerases includes alteration of integrin affinity states, facilitation of HIV-1 gp120 envelope protein cleavage before viral entry into CD4 T cells, and modulation of platelet adhesion. Moreover, calreticulin on the surface of dendritic cells functions as a receptor for tumor-associated NY-ESO-1 antigen. The results here demonstrated ERP5 function facilitates tumor immune evasion and may influence autoimmune diseases through sMICA-mediated T cell modulation.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### XI. REFERENCES

The following references, to the extent that they provide exemplary procedural or other details supplementary to those set forth herein, are specifically incorporated herein by reference.
U.S. Pat. No. 3,817,837
U.S. Pat. No. 3,850,752
U.S. Pat. No. 3,939,350
U.S. Pat. No. 3,996,345
U.S. Pat. No. 4,275,149
U.S. Pat. No. 4,277,437
U.S. Pat. No. 4,366,241
U.S. Pat. No. 4,683,195
U.S. Pat. No. 4,683,202
U.S. Pat. No. 4,725,669
U.S. Pat. No. 4,800,159
U.S. Pat. No. 4,845,026
U.S. Pat. No. 5,010,175
U.S. Pat. No. 5,068,174
U.S. Pat. No. 5,229,275
U.S. Pat. No. 5,288,514
U.S. Pat. No. 5,356,779
U.S. Pat. No. 5,506,337
U.S. Pat. No. 5,519,134
U.S. Pat. No. 5,525,735
U.S. Pat. No. 5,539,083
U.S. Pat. No. 5,549,974
U.S. Pat. No. 5,567,610
U.S. Pat. No. 5,569,588
U.S. Pat. No. 5,593,853
U.S. Pat. No. 5,840,873
U.S. Pat. No. 5,843,640
U.S. Pat. No. 5,843,650
U.S. Pat. No. 5,843,651
U.S. Pat. No. 5,843,663
U.S. Pat. No. 5,846,708
U.S. Pat. No. 5,846,709
U.S. Pat. No. 5,846,717
U.S. Pat. No. 5,846,726
U.S. Pat. No. 5,846,729
U.S. Pat. No. 5,846,783
U.S. Pat. No. 5,849,481
U.S. Pat. No. 5,849,486
U.S. Pat. No. 5,849,487
U.S. Pat. No. 5,849,497
U.S. Pat. No. 5,849,546
U.S. Pat. No. 5,849,547
U.S. Pat. No. 5,851,772
U.S. Pat. No. 5,853,990
U.S. Pat. No. 5,853,992
U.S. Pat. No. 5,853,993
U.S. Pat. No. 5,856,092
U.S. Pat. No. 5,858,652
U.S. Pat. No. 5,861,244
U.S. Pat. No. 5,863,732
U.S. Pat. No. 5,863,753
U.S. Pat. No. 5,866,331
U.S. Pat. No. 5,866,366
U.S. Pat. No. 5,882,864
U.S. Pat. No. 5,900,481
U.S. Pat. No. 5,905,024
U.S. Pat. No. 5,910,407
U.S. Pat. No. 5,912,124
U.S. Pat. No. 5,912,145
U.S. Pat. No. 5,916,776
U.S. Pat. No. 5,919,626
U.S. Pat. No. 5,919,630
U.S. Pat. No. 5,922,574
U.S. Pat. No. 5,925,517
U.S. Pat. No. 5,928,862
U.S. Pat. No. 5,928,869
U.S. Pat. No. 5,928,905
U.S. Pat. No. 5,928,906
U.S. Pat. No. 5,929,227
U.S. Pat. No. 5,932,413
U.S. Pat. No. 5,932,451
U.S. Pat. No. 5,935,791
U.S. Pat. No. 5,935,825
U.S. Pat. No. 5,939,291
U.S. Pat. No. 5,942,391
U.S. Pat. No. 6,040,152
U.S. Publn. 2001/0007666
U.S. Publn. 2005/0136102
U.S. Ser. No. 10/898,003
Abbondanzo, Ann. Diagn. Pathol., 3(5):318-327, 1999.
Akiyama et al., Alzheimer Dis. Assoc. Disord., 14(1):S47-53, 2000.
Allred et al., Arch. Surg., 125(1):107-113, 1990a.
Allred et al., Breast Cancer Res. Treat., 16:182(149), 1990b.
André et al., Eur. J. Immunol., 34:1-11, 2004.
Ausubel et al., In: Current Protocols in Molecular Biology, John, Wiley & Sons, Inc, NY, 1996.
Autenrieth et al., Infect. Immun., 62:2590-2599, 1994.
Bahram and Spies, Immunogenetics, 43(4):230-233, 1996.
Bahram et al., Proc. Natl. Acad. Sci. USA, 91(14):6259-6263, 1994.
Bauer et al., Science, 285(5428):727-729, 1999.
Baum, C & EN, 33, 1993.
Bendzen et al., Scand. J. Rheumatol., 28:599-606, 1988.
Blumberg et al., Arthritis Rheum., 7:93-97, 1964.
Brandt et al., Arthritis Rheum., 43:1346-1352, 2000.
Braun et al., Arthritis Rheum., 42:2039-2044, 1999.
Brown et al. Immunol. Ser., 53:69-82, 1990.
Bucknall et al., J. Am. Soc. Mass Spectrom., 13(9):1015-1027, 2002.
Campbell et al., J. Org. Chem., 59:658, 1994.
Cann et al., Gut., 24(12):1135-1140, 1983.
Capaldi et al., Biochem. Biophys. Res. Comm., 74(2):425-433, 1977.
Chen et al., J. Amer. Chem. Soc., 116:2661, 1994.
Chen et al., J. Chromatogr. B. Biomed. Sci. Appl., 755, 2000.
Cho et al., Science, 261:1303, 1993.
Chomarat et al., Arthritis Rheum., 38:1046-1054, 1995.
Das et al., Immunity, 15(1):83-93, 2001.
De Jager et al., Semin. Nucl. Med., 23(2):165-179, 1993.
de Waal et al., J. Exp. Med., 174:1209-1220, 1991.
Deckert et al., Int. J. Cancer, 87(3):382-90, 2000.
Diefenbach et al., Nat. Immunol., 3(12): 1142-1149, 2002.
Doolittle and Ben-Zeev, Methods Mol Biol, 109:215-237, 1999.
Drossman et al., Dig. Dis. Sci., 38(9):1569-1580, 1993.
Drossman et al., Gastroenterol., 112(6):2120-2137, 1997.
Eikelenboom et al., Glia, 40(2):232-239, 2002.
Ettehadi et al., Clin. Exp. Immunol., 96(1):146-151, 1994.
Everhart et al., Gastroenterol., 100(4):998-1005, 1991.
Furka, Int. J. Pept. Prot. Res., 37:487-493, 1991.
GB Appln. 2 202 328
Gehrmann et al., Neuropathol. Appl. Neurobiol., 21:277-289, 1995.
Gladman et al., J. Rheumatol., 22:675-679, 1995.
Gladman et al., Q. J. Med., 62:127-141, 1987.
Gladman, Rheum. Dis. Clin. North Am., 18:247-256, 1992.
Gobom et al., Anal. Chem., 72(14):3320-3326, 2000.
Graeber et al., Glia, 40(2):252-259, 2002.
Green et al., Nature Genet., 7:13, 1994.
Griffin et al., Proc. Natl. Acad. Sci. USA, 86(19):7611-7615, 1989.
Groh et al., Proc. Natl. Acad. Sci. USA, 98:6879, 1998.
Groh et al., Nat. Immun., 2(3): 255-260, 2001.
Groh et al., Nature, 419(6908):734-738, 2002.
Groh et al., Proc. Natl. Acad. Sci. USA, 100(16):9452-9457, 2003.
Groh et al., Proc. Natl. Acad. Sci. USA, 93:12445-12450, 1996.
Groh et al., Proc. Natl. Acad. Sci. USA, 96(12):6879-6884, 1999.
Grossberger and Parham, Immunogenetics, 36(3):166-174, 1992.
Gulbis and Galand, Hum. Pathol., 24(12):1271-1285, 1993.
Gwee et al., Gut., 44(3):400-406., 1999.
Hagihara et al., J. Amer. Chem. Soc., 114:6568, 1992.
Harris et al., Nat Rev Drug Discov., 2(3):214-21, 2003.
Harrison and Symmons et al., Ann. Rheum. Dis., 57(6):375-377, 1998.
Harrison et al., J. Rheumatol., 25(12):2324-2330, 1998.
Hart et al., Immunology, 84:536-542, 1995.
Hirschmann et al., J. Amer. Chem. Soc., 114:9217-9218, 1992.
Hobbs et al., Proc. Natl. Acad. Sci. USA, 90:6909-6913, 1993.
Hohler et al., Arthritis Rheum., 41:1489-1492, 1998.
Hohler et al., J. Invest. Dermatol., 109:562-565, 1997.
Horwitz and Fisher, N. Engl. J. Med., 344(24):1846-1850, 2001.
Houghton et al., Nature, 354:84-88, 1991.
Innis et al., Proc. Natl. Acad. Sci. USA, 85(24):9436-9440, 1988.
Ishizawa and Dickson, J Neuropathol Exp Neurol. 60:647-657, 2001.
Jacob et al., Proc. Natl. Acad. Sci. USA, 87:1233-1237, 1990.
Jailwala et al., Ann. Intern. Med., 133(2):136-147, 2000.
Jones et al., Br. J. Rheumatol., 33:834-839, 1994.
Jordan et al., Blood, 105(4):1500-1507, 2005.
Kellow and Phillips, Gastroenterol., 92(6):1885-1893, 1987.
Kotake et al., Infect. Immun., 67:2682-2686, 1999.
Lahesmaa et al., J. Immunol., 148:3079-3085, 1992.
Lee et al., Bioconjug Chem., 14(3):546-53, 2003.
Li et al., Immunity, 10: 577-584, 1999.
Li et al., Nat. Immunol., 2: 443-451, 2001.
Liang et al., Science, 274:1520-1522, 1996.
Lonberg et al., Nature, 368:856, 1994.
Lynn and Friedman, N. Engl. J. Med., 329(26):1940-1945, 1993.
Macatonia et al., J. Immunol., 150:3755-3765, 1993.
Marsal et al., Rheumatology, 38:332-337, 1999.
McCafferty et al., Nature, 348:552-553, 1990.
McGonagle et al., Arthritis Rheum., 41:694-700, 1998.
McGonagle et al., Curr. Opin. Rheumatol., 11:244-250, 1999.
Mease et al., Lancet, 356:385-390, 2000.
Mertz et al., Gastroenterol., 118(5):842-848, 2000.
Mirgorodskaya et al., Rapid Commun. Mass Spectrom., 14(14):1226-1232, 2000.
Moll and Wright, Ann. Rheum. Dis., 32:181-201, 1973.
Moll and Wright, Semin. Arthritis Rheum., 3:55-78, 1973.
Nakamura et al., In: Handbook of Experimental Immunology (4^{th} Ed.), Weir et al. (Eds.), 1:27, Blackwell Scientific Publ., Oxford, 1987.
Neal et al., BMJ, 314(7083):779-782, 1997.
Nielen et al., Arthritis Rheum., 50(2):380-386, 2004.
OMIM 602893
OMIM 604089
OMIM 604142
Orr et al., Progr Neurobiol., 68:325-340, 2002.
Partsch et al., Br. J. Rheumatol., 24:518-523, 1997.
PCT Appln. PCT/US03/12299
PCT Appln. PCT/US89/01025
PCT Appln. PCT/US96/10287
PCT Appln. WO 90/07641
PCT Appln. WO 91/19735
PCT Appln. WO 92/00091
PCT Appln. WO 93/20242
Pimentel et al., Am. J. Gastroenterol., 95(12):3503-3506, 2000.
Pociot et al., Scand. J. Immunol., 42(4):501-504, 1995.
Rantapaa-Dahlqvist et al., Arthritis Rheum., 48(10):2741-2749, 2003.
Remington's Pharmaceutical Sciences, 15^{th} ed., 33:624-652, Mack Publishing Company, Easton, Pa., 1980.
Ribbens et al., Eur. Cytokine Netw., 11:669-676, 2000.
Rogers et al., Neurobiol Aging, 9(4):339-349, 1988.
Rothstein, Med. Clin. North Am., 84(5):1247-1257, 2000.
Salvarani et al., Curr. Opin. Rheumatol., 10:299-305, 1998.
Sambrook et al., In: Molecular cloning: a laboratory manual, 2^{nd} Ed., Cold Spring Harbor Laboratory Press, Cold Spring Harbor, N.Y., 1989.
Sandler, Gastroenterol., 99(2):409-415, 1990.
Schellekens et al., Arthritis Rheum., 43(1):155-163, 2000.
Schlaak et al., Clin. Exp. Rheumatol., 14:155-162, 1996.
Schlaak et al., Eur. J. Immunol., 22:2771-2776, 1992.
Sieper and Braun, Arthritis Rheum., 38:1547-1554, 1995.
Simon et al., Bio/Technology, 1:784-791, 1983.
Simon et al., Proc. Natl. Acad. Sci. USA, 91:8562-85666, 1994.
Spies et al., Proc. Natl. Acad. Sci. USA, 86(22):8955-8958, 1989.
Steinle et al., Immunogenetics, 53(4):279-87, 2001.
Stroynowski, Annu Rev Immunol., 8:501-530, 1990.
Talley et al., Gastroenterology, 109(6): 1736-1741, 1995.
Taylor et al., Int. Immun., 6:579, 1994.
Tieng et al., Proc. Natl. Acad. Sci. USA, 99(5):2977-82, 2002.
Touzani et al., J. Neuroimmunol., 100(1-2):203-215, 1999.
Vaughn et al., Nature Biotechnology, 14(3):309-314, 1996.
Whitehead et al., Gastroenterol., 98(5 Pt 1):1187-1192, 1990.
Wright, Ann. Rheum. Dis., 15:348-356, 1956.
Wright, Clin. Orthop. Related Res., 143:8-14, 1979.
Wu et al., Anal. Chem., 72:61, 2000.
Wu et al., Science, 285(5428):730-732, 1999.
Yin et al., Arthritis Rheum., 40:1788-1797, 1997.
Yin et al., Rheumatology, 38:1058-1067, 1999.
Zhong et al., Clin. Chem. ACTA., 313:147, 2001.

What is claimed is:

1. A method of screening for an immunomodulatory agent comprising:
(a) providing a cell that expresses ERP5 and MICA/MICB;
(b) culturing the cell with a candidate substance; and
(c) assessing one or more of (i) cell surface expression of MICA/MICB, (ii) MICA/MICB complexing with ERP5, (iii) disulfide bond reduction in MICA/MICB, (iv) MICA/MICB tetramer binding, (v) proteolytic cleavage of MICA/MICB, (vi) presence of soluble MICA/MICB, (vii) ERP5 transcription, translation or cell surface expression, and/or (viii) MICA/MICB autoantibodies,
wherein a change in any of (c)(i)-(vii), as compared to a cell not treated with the candidate substance, identifies the candidate substance as an immunomodulatory agent.

2. The method of claim 1, wherein the candidate substance is a peptide, protein, an RNA, a DNA, an organopharmaceutical, or a lipid.

3. The method of claim 1, wherein the cell is an epithelial tumor cell, activated lymphocyte, synoviocyte, leukemia cell, activated hematopoietic cell, inflamed cell, infected cell or a cell derived from an autoimmune lesion.

4. A method of modulating MICA/MICB cleavage in a cell that expresses MICA/MICB and ERP5 comprising contacting the cell with a modulator of ERP5 expression or function.

5. The method of claim 4, wherein the modulator alters ERP5 release of MICA/MICB.

6. The method of claim 4, wherein the modulator alters ERP5 binding to MICA/MICB.

7. The method of claim 4, wherein the modulator alters ERP5 isomerization of MICA/MICB.

8. The method of claim 4, wherein the modulator alters ERP5 thioreduction of MICA/MICB.

9. The method of claim 4, wherein the modulator alters ERP5 transcription or translation or cell surface expression.

10. The method of claim 4, wherein the cell is an epithelial tumor cell, activated lymphocyte, synoviocyte, leukemia cell, activated hematopoietic cell, inflamed cell, infected cell or a cell derived from an autoimmune lesion.

11. The method of claim 4, wherein the modulator is an antagonist that is a competing substrate for EPR5.

12. The method of claim 4, wherein the modulator is an antagonist that is a thioreductase inhibitor.

13. The method of claim 12, wherein the modulator is an antagonist selected from bacitracin, DTNB and PAO.

14. A method of reducing MICA and/or MICB cleavage by ERP5 in a subject comprising:
(a) selecting a binding agent on the basis of its ability to bind MICA/MICB and block cleavage of MICA and/or MICB by ERP5;
(b) admininstering said binding agent to said subject in an amount and route sufficient to reduce MICA and/or MICA cleavage by ERP5.

15. The method of claim 14, wherein said binding agent is antisera or a monoclonal antibody.

16. The method of claim 14, wherein said binding agent comprises a non-cleaving form of ERP5.

17. The method of claim 14, wherein said subject has cancer.

18. The method of claim 14, wherein said binding agent does not block MICA and/or MICB binding to NKG2D.

19. The method of claim 14, wherein said binding agent binds at or near the ERP5 cleavage site in MICA and/or MICB.

20. The method of claim 14, wherein said binding agent binds distal to the ERP5 cleavage site and cause a conformational change such that ERP5 does not cleave yjr MICA and/or MICB.

21. A method of treating a subject with an autoimmune disease comprising administering to the subject an agonist of an NKG2D⁺ CD4⁺ cell.

22. The method of claim 21, wherein said agonist is MICA, MICB, anti-NKG2D⁺ antibody or derivative thereof, DAP10 or ULB1-10.

23. The method of claim 21, wherein the autoimmune disease may be RA, SLE, juvenile SLE, sclerodema, MS, Crohn's disease, celiac disease, inflammatory bowel disease, rheumatoid arthritis, insulin-dependent diabetes mellitus (type 1), multiple sclerosis, Wegener's granulomatosis, Sjogren's syndrome, systemic lupus erythematosus, myasthenia gravis, Reiter's syndrome, Grave's disease, Hashimoto's thyroiditis, pernicious anemia, Addison's disease, dermatomyositis, polymyositis, T-cell mediated transplant rejection and Guillain Barré.

24. A method of treating a subject with an MIC-secreting tumor comprising (a) assessing MIC secretion by the tumor and (b) administering to said subject an antagonist of FasL.

25. A method of treating a subject with an autoimmune disease comprising administering to said subject an agonist of FasL activity.

26. The method of claim 25, wherein the autoimmune disease is RA, SLE, sclerodema, MS, Crohn's disease, celiac disease, inflammatory bowel disease, rheumatoid arthritis, insulin-dependent diabetes mellitus (type 1), multiple sclerosis, Wegener's granulomatosis, Sjogren's syndrome, systemic lupus erythematosus, myasthenia gravis, Reiter's syndrome, Grave's disease, Hashimoto's thyroiditis, pernicious anemia, Addison's disease, dermatomyositis, polymyositis, GVHD and Guillain Barré.


SEQUENCE LISTING (12 sequences)

SEQ ID NO: 1 - DNA, 1365 bp, Homo sapiens
  CDS (40)..(1191)
cactgcttgagccgctgagagggtggcgacgtcggggccatggggctgggcccggtcttcctgcttctggctggcatcttcccttttgcacctccgggagctgctgctgagccccacagtcttcgttataacctcacggtgctgtcctgggatggatctgtgcagtcagggtttctcactgaggtacatctggatggtcagcccttcctgcgctgtgacaggcagaaatgcagggcaaagccccagggacagtgggcagaagatgtcctgggaaataagacatgggacagagagaccagagacttgacagggaacggaaaggacctcaggatgaccctggctcatatcaaggaccagaaagaaggcttgcattccctccaggagattagggtctgtgagatccatgaagacaacagcaccaggagctcccagcatttctactacgatggggagctcttcctctcccaaaacctggagactaaggaatggacaatgccccagtcctccagagctcagaccttggccatgaacgtcaggaatttcttgaaggaagatgccatgaagaccaagacacactatcacgctatgcatgcagactgcctgcaggaactacggcgatatctaaaatccggcgtagtcctgaggagaacagtgccccccatggtgaatgtcacccgcagcgaggcctcagagggcaacattaccgtgacatgcagggcttctggcttctatccctggaatatcacactgagctggcgtcaggatggggtatctttgagccacgacacccagcagtggggggatgtcctgcctgatgggaatggaacctaccagacctgggtggccaccaggatttgccaaggagaggagcagaggttcacctgctacatggaacacagcgggaatcacagcactcaccctgtgccctctgggaaagtgctggtgcttcagagtcattggcagacattccatgtttctgctgttgctgctgctgctatttttgttattattattttctatgtccgttgttgtaagaagaaaacatcagctgcagagggtccagagctcgtgagcctgcaggtcctggatcaacacccagttgggacgagtgaccacagggatgccacacagctcggatttcagcctctgatgtcagatcttgggtccactggctccactgagggcgcctagactctacagccaggcagctgggattcaattccctgcctggatctcacgagcactttccctcttggtgcctcagtttcctgacctatgaaacagagaaaataaaagcacttatttattgttgttggaggctgcaaaatgttagtagatatgaggcgtttgcagctgtaccatatt

SEQ ID NO: 2 - protein, 383 aa, Homo sapiens
MGLGPVFLLLAGIFPFAPPGAAAEPHSLRYNLTVLSWDGSVQSGFLTEVHLDGQPFLRCDRQKCRAKPQGQWAEDVLGNKTWDRETRDLTGNGKDLRMTLAHIKDQKEGLHSLQEIRVCEIHEDNSTRSSQHFYYDGELFLSQNLETKEWTMPQSSRAQTLAMNVRNFLKEDAMKTKTHYHAMHADCLQELRRYLKSGVVLRRTVPPMVNVTRSEASEGNITVTCRASGFYPWNITLSWRQDGVSLSHDTQQWGDVLPDGNGTYQTWVATRICQGEEQRFTCYMEHSGNHSTHPVPSGKVLVLQSHWQTFHVSAVAAAAIFVIIIFYVRCCKKKTSAAEGPELVSLQVLDQHPVGTSDHRDATQLGFQPLMSDLGSTGSTEGA

SEQ ID NO: 3 - DNA, 2385 bp, Homo sapiens
  CDS (6)..(1157)
gggccatggggctgggccgggtcctgctgtttctggccgtcgccttcccttttgcacccccggcagccgccgctgagccccacagtcttcgttacaacctcatggtgctgtcccaggatggatctgtgcagtcagggtttctcgctgagggacatctggatggtcagcccttcctgcgctatgacaggcagaaacgcagggcaaagccccagggacagtgggcagaagatgtcctgggagctgagacctgggacacagagaccgaggacttgacagagaatgggcaagacctcaggaggaccctgactcatatcaaggaccagaaaggaggcttgcattccctccaggagattagggtctgtgagatccatgaagacagcagcaccaggggctcccggcatttctactacaatggggagctcttcctctcccaaaacctggagactcaagaatcgacagtgccccagtcctccagagctcagaccttggctatgaacgtcacaaatttctggaaggaagatgccatgaagaccaagacacactatcgcgctatgcaggcagactgcctgcagaaactacagcgatatctgaaatccggggtggccatcaggagaacagtgccccccatggtgaatgtcacctgcagcgaggtctcagagggcaacatcaccgtgacatgcagggcttccagcttctatccccggaatatcacactgacctggcgtcaggatggggtatctttgagccacaacacccagcagtggggggatgtcctgcctgatgggaatggaacctaccagacctgggtggccaccaggattcgccaaggagaggagcagaggttcacctgctacatggaacacagcgggaatcacggcactcaccctgtgccctctgggaaggcgctggtgcttcagagtcaacggacagactttccatatgtttctgctgctatgccatgttttgttattattattattctctgtgtcccttgttgcaagaagaaaacatcagcggcagagggtccagagcttgtgagcctgcaggtcctggatcaacacccagttgggacaggagaccacagggatgcagcacagctgggatttcagcctctgatgtcagctactgggtccactggttccactgagggcgcctagactctacagccaggcggccaggattcaactccctgcctggatctcaccagcactttccctctgtttcctgacctatgaaacagaaaataacatcacttatttattgttgttggatgctgcaaagtgttagtaggtatgaggtgtttgctgctctgccacgtagagagccagcaaagggatcatgaccaactcaacattccattggaggctatatgatcaaacagcaaattgtttatcatgaatgcaggatgtgggcaaactcacgactgctcctgccaacagaaggtttgctgagggcattcactccatggtgctcattggagttatctactgggtcatctagagcctattgtttgaggaatgcagtcttacaagcctactctggacccagcagctgactccttcttccacccctcttcttgctatctcctataccaataaatacgaagggctgtggaagatcagagcccttgttcacgagaagcaagaagccccctgaccccttgttccaaatatactcttttgtctttctctttattcccacgttcgccctttgttcagtccaatacagggttgtggggcccttaacagtgccatattaattggtatcattatttctgttgtttttgtttttgtttttgtttttgtttttgagacagagtctcactctgtcacccaggctgcagttcactggtgtgatctcagctcactgcaacctctgcctcccaggttcaagcacttctcgtacctcagactcccgaatagctgggattacagacaggcaccaccacacccagctaatttttgtattttttgtagagacggggtttcgccaagttgaccagcccagtttcaaactcctgacctcaggtgatctgcctgccttggcatcccaaagtgctgggattacaagaatgagccaccgtgcctggcctattttattatattgtaatatattttattatattagccaccatgcctgtcctattttcttatgttttaatatattttaatatattacatgtgcagtaattagattatcatgggtgaactttatgagtgagtatcttggtgatgactcctcctgaccagcccaggaccagctttcttgtcaccttgaggtcccctcgccccgtcacaccgttatgcattactctgtgtctactattatgtgtgcataatttataccgtaaatgtttactctttaaatagaaaaaaaaaaaaaaaa

SEQ ID NO: 4 - protein, 383 aa, Homo sapiens
MGLGRVLLFLAVAFPFAPPAAAAEPHSLRYNLMVLSQDGSVQSGFLAEGHLDGQPFLRYDRQKRRAKPQGQWAEDVLGAETWDTETEDLTENGQDLRRTLTHIKDQKGGLHSLQEIRVCEIHEDSSTRGSRHFYYNGELFLSQNLETQESTVPQSSRAQTLAMNVTNFWKEDAMKTKTHYRAMQADCLQKLQRYLKSGVAIRRTVPPMVNVTCSEVSEGNITVTCRASSFYPRNITLTWRQDGVSLSHNTQQWGDVLPDGNGTYQTWVATRIRQGEEQRFTCYMEHSGNHGTHPVPSGKALVLQSQRTDFPYVSAAMPCFVIIIILCVPCCKKKTSAAEGPELVSLQVLDQHPVGTGDHRDAAQLGFQPLMSATGSTGSTEGA

SEQ ID NO: 5 - DNA, 66 bp, Artificial Sequence
  Description of Artificial Sequence  Synthetic Primer
gatcttgttgtcaaagttggtgcagttgtcttcttctcaactgcaccaactttgacaacatttttg

SEQ ID NO: 6 - DNA, 66 bp, Artificial Sequence
  Description of Artificial Sequence  Synthetic Primer
aattcaaaaatgttgtcaaagttggtgcagttgagaagaagacaactgcaccaactttgacaacaa

SEQ ID NO: 7 - DNA, 66 bp, Artificial Sequence
  Description of Artificial Sequence  Synthetic Primer
gatcttgatagttcaagtaagaaggatgtcttcttctcatccttcttacttgaactatcatttttg

SEQ ID NO: 8 - DNA, 66 bp, Artificial Sequence
  Description of Artificial Sequence  Synthetic Primer
aattcaaaaatgatagttcaagtaagaaggatgagaagaagacatccttcttacttgaactatcaa

SEQ ID NO: 9 - DNA, 19 bp, Artificial Sequence
  Description of Artificial Sequence  Synthetic Primer
tgcggcacgctgcagggct

SEQ ID NO: 10 - DNA, 27 bp, Artificial Sequence
  Description of Artificial Sequence  Synthetic Primer
ttgacagtgaccacaccatggagcata

SEQ ID NO: 11 - DNA, 23 bp, Artificial Sequence
  Description of Artificial Sequence  Synthetic Primer
ggaacggaaaggacctcaggatg

SEQ ID NO: 12 - DNA, 22 bp, Artificial Sequence
  Description of Artificial Sequence  Synthetic Primer
ctgggagctcctggtgctgttg